(19) United States
(12) Patent Application Publication
(10) Pub. No.: US 2026/0337154 A1
(43) Pub. Date: Oct. 29, 2026

(54) ZWITTERIONIC SULFOBETAINE-CONTAINING LIPIDS AND USES THEREOF

(71) Applicant: Bausch + Lomb Ireland Limited, Dublin (IE)

(72) Inventor: Jamie Homnick, Webster, NY (US)

(21) Appl. No.: 19/649,558

(22) Filed: Apr. 16, 2026

Related U.S. Application Data
(60) Provisional application No. 63/790,256, filed on Apr. 17, 2025.

(51) Int. Cl. C07C 309/21; G02B 1/04

(52) U.S. Cl. CPC C07C 309/21; G02B 1/043

(57) ABSTRACT

A zwitterionic sulfobetaine-containing lipid includes a zwitterionic sulfobetaine group attached to one or more lipid groups each comprising at least one fatty acid acyl chain.

### PRIORITY CLAIM

The present application claims priority to U.S. Provisional Patent Application Ser. No. 63/790,256, entitled “Sulfobetaine Zwitterionic-Containing Lipids and Uses Thereof,” filed Apr. 17, 2025, the content of which is incorporated by reference herein in its entirety.

### BACKGROUND

It is highly desirable that a contact lens be as comfortable as possible for wearers. Manufacturers of contact lenses are continually working to improve the comfort of the lenses. Nevertheless, many people who wear contact lenses still experience dryness or eye irritation throughout the day and particularly towards the end of the day. An insufficiently wetted lens at any point in time will cause significant discomfort to the lens wearer. Although wetting drops can be used as needed to alleviate such discomfort, it would certainly be desirable if such discomfort did not arise in the first place.

### SUMMARY

In accordance with an illustrative embodiment, a zwitterionic sulfobetaine-containing lipid comprises a zwitterionic sulfobetaine group attached to one or more lipid groups each comprising at least one fatty acid acyl chain.

In accordance with another illustrative embodiment, an ophthalmic device comprises a zwitterionic sulfobetaine-containing lipid entrapped in the ophthalmic device, wherein the zwitterionic sulfobetaine-containing lipid comprises a zwitterionic sulfobetaine group attached to one or more lipid groups each comprising at least one fatty acid acyl chain.

In accordance with another illustrative embodiment, a process for preparing an ophthalmic device having a zwitterionic sulfobetaine-containing lipid entrapped in the ophthalmic device comprises:
subjecting an ophthalmic device-forming mixture comprising one or more ophthalmic device-forming monomers and a zwitterionic sulfobetaine-containing lipid comprising a zwitterionic sulfobetaine group attached to one or more lipid groups each comprising at least one fatty acid acyl chain to polymerization conditions to provide a polymerized ophthalmic device, and
hydrating the polymerized ophthalmic device.

In accordance with another illustrative embodiment, a process for preparing an ophthalmic device having a zwitterionic sulfobetaine-containing lipid entrapped in the ophthalmic device comprises:
soaking an ophthalmic device in one or more first solvent solutions to swell the ophthalmic device, thereby providing a swelled ophthalmic device,
soaking the swelled ophthalmic device in one or more second solvent solutions to sulfamethazine de-swell the ophthalmic device, thereby providing a de-swelled ophthalmic device, and
sterilizing the de-swelled ophthalmic device,
wherein the one or more first solvent solutions, the one or more second solvent solutions, or both comprise a zwitterionic sulfobetaine-containing lipid comprising a zwitterionic sulfobetaine group attached to one or more lipid groups each comprising at least one fatty acid acyl chain.

In accordance with another illustrative embodiment, a packaging system for the storage of an ophthalmic device comprises a sealed container containing an unused ophthalmic device immersed in an aqueous packaging solution comprising a zwitterionic sulfobetaine-containing lipid comprising a zwitterionic sulfobetaine group attached to one or more lipid groups each comprising at least one fatty acid acyl chain, wherein the aqueous packaging solution has an osmolality of at least about 200 mOsmol/kg, a pH of about 6 to about 9 and is sterilized.

In accordance with another illustrative embodiment, a method of preparing a package comprising a storable, sterile ophthalmic device comprises:
immersing an unused ophthalmic device in an aqueous packaging solution comprising a zwitterionic sulfobetaine-containing lipid comprising a zwitterionic sulfobetaine group attached to one or more lipid groups each comprising at least one fatty acid acyl chain, wherein the aqueous packaging solution has an osmolality of at least about 200 mOsmol/kg and a pH in the range of about 6 to about 9,
packaging the aqueous packaging solution and the unused ophthalmic device in a manner preventing contamination of the device by microorganisms, and
sterilizing the packaged aqueous packaging solution and the unused ophthalmic device.

In accordance with another illustrative embodiment, an aqueous ophthalmic composition comprises a zwitterionic sulfobetaine-containing lipid comprising a zwitterionic sulfobetaine group attached to one or more lipid groups each comprising at least one fatty acid acyl chain, wherein the aqueous ophthalmic composition has an osmolality in a range from about 200 mOsmol/kg to about 400 mOsmol/kg.

### BRIEF DESCRIPTION OF THE DRAWINGS

FIGS. 1A and 1B illustrate a zwitterionic sulfobetaine-containing lipid in an alternating pattern with a phospholipid to form a two-dimensional alternating coulombically associated network, according to one or more illustrative embodiments.

### DETAILED DESCRIPTION

The illustrative embodiments described herein are directed to zwitterionic sulfobetaine-containing lipid useful in treating the surface of an ophthalmic device intended for direct contact with body tissue or fluid.

### DEFINITIONS

To define more clearly the terms used herein, the following definitions are provided. Unless otherwise indicated, the following definitions are applicable to this disclosure. If a term is used in this disclosure but is not specifically defined herein, the definition from the IUPAC Compendium of Chemical Terminology can be applied, as long as that definition does not conflict with any other disclosure or definition applied herein or render indefinite or non-enabled any claim to which that definition is applied. To the extent that any definition or usage provided by any document incorporated herein by reference conflicts with the definition or usage provided herein, the definition or usage provided herein controls.

While compositions and processes are described in terms of “comprising” various components or steps, the compositions and processes can also “consist essentially of” or “consist of” the various components or steps, unless stated otherwise.

The terms “a,” “an,” and “the” are intended to include plural alternatives, e.g., at least one. The terms “including,” “with,” and “having,” as used herein, are defined as comprising (i.e., open language), unless specified otherwise.

Various numerical ranges are disclosed herein. When Applicant discloses or claims a range of any type, Applicant's intent is to disclose or claim individually each possible number that such a range could reasonably encompass, including end points of the range as well as any sub-ranges and combinations of sub-ranges encompassed therein, unless otherwise specified. For example, all numerical end points of ranges disclosed herein are approximate, unless excluded by proviso.

Values or ranges may be expressed herein as “about,” from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself. In another aspect, use of the term “about” means±20% of the stated value, ±15% of the stated value, ±10% of the stated value, ±5% of the stated value, ±3% of the stated value, or 11% of the stated value.

The terms “wt. %,” “vol. %” or “mol. %” refers to a weight, volume, or molar percentage of a component, respectively, based on the total weight, the total volume, or the total moles of material that includes the component. In a non-limiting example, 10 moles of component in 100 moles of the material are 10 mol. % of component.

Applicant reserves the right to proviso out or exclude any individual members of any such group of values or ranges, including any sub-ranges or combinations of sub-ranges within the group, that can be claimed according to a range or in any similar manner, if for any reason Applicant chooses to claim less than the full measure of the disclosure, for example, to account for a reference that Applicant may be unaware of at the time of the filing of the application. Further, Applicant reserves the right to proviso out or exclude any members of a claimed group.

As used herein, the term “ophthalmic device” refers to devices that reside in or on the eye. These ophthalmic devices can provide optical correction, wound care, drug delivery, diagnostic functionality or cosmetic enhancement or effect or a combination of these properties. Representative examples of such devices include, but are not limited to, soft contact lenses, e.g., a soft, hydrogel lens; soft, non-hydrogel lens and the like, hard contact lenses, e.g., a hard, gas permeable lens material and the like, intraocular lenses, overlay lenses, ocular inserts, optical inserts and the like. As is understood by one skilled in the art, a lens is considered to be “soft” if it can be folded back upon itself without breaking. A contact lens can be in a dry state or a wet state. A “dry state” refers to a soft contact lens in a state prior to hydration or the state of a hard lens under storage or use conditions. A “wet state” refers to a soft contact lens in a hydrated state.

The eye produces tears that are spread across the eye while blinking. The unique components of tears combined with the blinking process create a tear film that normally includes a three-layer structure: a lipid layer, a middle aqueous layer, and a mucinous layer. The lipid layer is the outermost layer and derived from the secretions of the Meibomian glands. It is likely composed of two phases: a thin polar phase adjacent to the aqueous-mucinous phase and a thick nonpolar phase associated with both the polar phase and the air interface. The middle aqueous layer is provided by the major and minor lacrimal glands, and contains water-soluble substances. The innermost mucinous layer is composed of glycoprotein, mucin, and overlies the corneal and conjunctival epithelial cells. The epithelial cell membranes are composed of lipoproteins and thus generally hydrophobic.

Contact lenses intrinsically disrupt the native tear film. In addition, the tear film undergoes significant forces that can compromise the integrity of the film including: (1) evaporation, (2) spreading along the ocular surface, which is driven by high shear blinks, (3) draining, which is aided by blink powered lacrimal pumping and (4) low shear flow along the lid tear menisci. To maintain the tear film, the tear film is continually replenished with new tear film components upon each blink, which is triggered by tear breakup and corneal surface nerve excitation. When any of the tear film components is deficient, the tear film will break up, and dry spots will form on the corneal and the conjunctival epithelium. A deficiency of any of the three components (aqueous, mucin or lipid layers) may result in dryness of the eye.

The non-limiting illustrative embodiments disclosed herein overcome the foregoing drawbacks by providing a zwitterionic sulfobetaine-containing lipid comprising a zwitterionic sulfobetaine attached to one or more lipid groups each comprising at least one fatty acid acyl chain which may advantageously provide improved lubricity to the surface of an ophthalmic device such as a contact lens while inhibiting or preventing dry eye. For example, the benefits of improved lubricity using a zwitterionic sulfobetaine-containing lipid comprising a zwitterionic sulfobetaine group attached to one or more lipid groups each comprising at least one fatty acid acyl chain of the present disclosure include, for example, strengthening and stabilizing the tear film lipid layer and alleviate the dryness of the eye, as well as reduced deposition (e.g., protein, lipid, etc.) and thus potentially reducing biofilm formation by the contact lens wearer onto the lens surface.

In accordance with a non-limiting illustrative embodiment, a zwitterionic sulfobetaine-containing lipid includes a zwitterionic sulfobetaine group attached to one or more lipid groups each comprising at least one fatty acid acyl chain. In some embodiments, a zwitterionic sulfobetaine-containing lipid can be represented by the following structure:

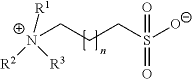

wherein R¹ is a hydrocarbyl group, R² is a hydrocarbyl group or one or more lipid groups each comprising at least one fatty acid acyl chain and R³ is a hydrocarbyl group or one or more lipid groups comprising at least one fatty acid acyl chain, wherein at least one of R² or R³ is one or more lipid groups each comprising at least one fatty acid acyl chain, and n is 1 or 2.

Representative examples of hydrocarbyl groups include linear or branched alkyl groups, linear or branched alkenyl groups, linear or branched alkynyl groups, cycloalkyl groups, heterocycloalkyl groups, aryl groups (including polycyclic aryl groups), heteroaryl groups (having at least one hetero atom in the aromatic ring); and aralkyl groups as defined herein.

As used herein, recitations of “linear or branched” groups, such as linear or branched alkyl, are herein understood to include, for example, groups that are linear, such as linear C₂ to C₃₀ alkyl groups; and groups that are appropriately branched, such as branched C₃ to C₃₀ alkyl groups.

Representative examples of alkyl groups for use herein include, by way of example, a linear or branched hydrocarbon chain radical containing carbon and hydrogen atoms of from 1 to about 30 carbon atoms or from 1 to 12 carbon atoms or from 1 to 6 carbon atoms with or without unsaturation, to the rest of the molecule, e.g., methyl, ethyl, n-propyl, 1-methylethyl (isopropyl), n-butyl, n-pentyl, etc., and the like.

Representative examples of alkenyl groups for use herein include, by way of example, a straight or branched hydrocarbon chain radical containing from about 3 to about 30 carbon atoms with at least one carbon-carbon double bond such as, for example, propenyl, butenyl, pentenyl and the like.

Representative examples of alkynyl groups for use herein include, by way of example, a straight or branched hydrocarbon chain radical containing from about 3 to about 30 carbon atoms with at least one carbon-carbon triple bond such as, for example, propynyl, butynyl, pentynyl and the like.

Representative examples of cycloalkyl groups for use herein include, by way of example, a substituted or unsubstituted non-aromatic mono or multicyclic ring system of about 3 to about 30 carbon atoms or from 3 to 12 carbon atoms or from 3 to 6 carbon atoms such as, for example, cyclopropyl, cyclobutyl, cyclopentyl, cyclohexyl, perhydronapththyl, adamantyl and norbornyl groups bridged cyclic group or sprirobicyclic groups, e.g., sprio-(4, 4)-non-2-yl and the like, optionally containing one or more heteroatoms, e.g., O and N, and the like.

Representative examples of heterocyclic groups for use herein include, by way of example, a substituted or unsubstituted stable 3 to about 15 membered ring radical, containing carbon atoms and from one to five heteroatoms, e.g., nitrogen, phosphorus, oxygen, sulfur and mixtures thereof. Suitable heterocyclic ring radicals for use herein may be a monocyclic, bicyclic or tricyclic ring system, which may include fused, bridged or spiro ring systems, and the nitrogen, phosphorus, carbon, oxygen or sulfur atoms in the heterocyclic ring radical may be optionally oxidized to various oxidation states.

Representative examples of aryl groups for use herein include, by way of example, a substituted or unsubstituted monoaromatic or polyaromatic radical containing from about 5 to about 30 carbon atoms or from 6 to 12 carbon atoms or from 6 to 8 carbon atoms such as, for example, phenyl, naphthyl, tetrahydronapthyl, indenyl, biphenyl and the like.

Representative examples of heteroaryl groups for use herein include, by way of example, a substituted or unsubstituted stable 5 to about 30 membered monoaromatic or polyaromatic radical, containing carbon atoms and from one to five heteroatoms, e.g., nitrogen, phosphorus, oxygen, sulfur and mixtures thereof.

Representative examples of aralkyl groups as used herein, and in accordance with some embodiments, include, but are not limited to, C₆ to C₂₄ aralkyl, such as a C₆ to C₁₀ aralkyl, and means an aryl group substituted with an alkyl group.

The lipid groups can be natural or synthetic. For example, a lipid can include esterified fatty acid acyl chains, or organic chains attached by non-ester linkages such as amide linkages. In some embodiment the lipid chains include from about 6 to about 30 carbon atoms, or from about 12 to about 26 carbon atoms, and can be saturated or unsaturated. Suitable fatty acid acyl chains include, for example, n-tetradecanoic, n-hexadecanoic acid, n-octadecanoic acid, n-eicosanoic acid, n-docosanoic acid, n-tetracosanoic acid, n-hexacosanoic acid, linoleic acid, arachidonic acid, and the like.

In some embodiments, an acyl moiety of the fatty acid acyl chain can be an ester group. In some embodiments, an acyl moiety of the fatty acid acyl chain can be an amide group. In some embodiments, an acyl moiety of the fatty acid acyl chain of the one or more lipid groups can be attached to the zwitterionic sulfobetaine moiety by way of an alkylene group, e.g., a C₁ to C₆ alkylene group.

In some embodiments, a zwitterionic sulfobetaine-containing lipid can be represented by the following structure:

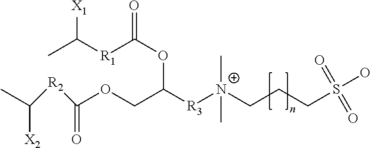

wherein R₁ is a saturated or unsaturated linear diradical C₁₃ to C₂₇ hydrocarbon chain,
R₂ is a saturated or unsaturated linear diradical C₁₃ to C₂₇ hydrocarbon chain,
R₃ is an alkylene group, preferably methylene,
X₁ and X₂ are independently H or CH₃, and
n is 1 or 2.

In some embodiments, a zwitterionic sulfobetaine-containing lipid can be represented by the following structure:

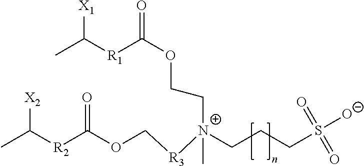

wherein R₁ is a saturated or unsaturated linear diradical C₁₃ to C₂₇ hydrocarbon chain,
R₂ is a saturated or unsaturated linear diradical C₁₃ to C₂₇ hydrocarbon chain,
R₃ is an alkylene group, preferably methylene,
X₁ and X₂ are independently H or CH₃, and
n is 1 or 2.

In some embodiments, a zwitterionic sulfobetaine-containing lipid can be represented by the following structure:

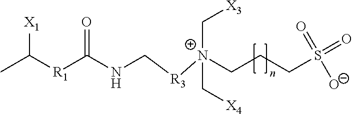

wherein R₁ is a saturated or unsaturated linear diradical C₁₃ to C₂₇ hydrocarbon chain,
R₃ is an alkylene group, preferably methylene,
X₁, X₃, and X₄ are independently H or CH₃, and
n is 1 or 2.

In one embodiment, a zwitterionic sulfobetaine-containing lipid can be obtained as set forth below in scheme I:

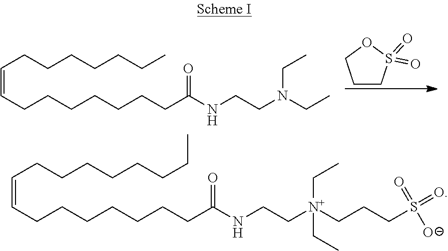

In one embodiment, a zwitterionic sulfobetaine-containing lipid can be obtained as set forth below in scheme II:

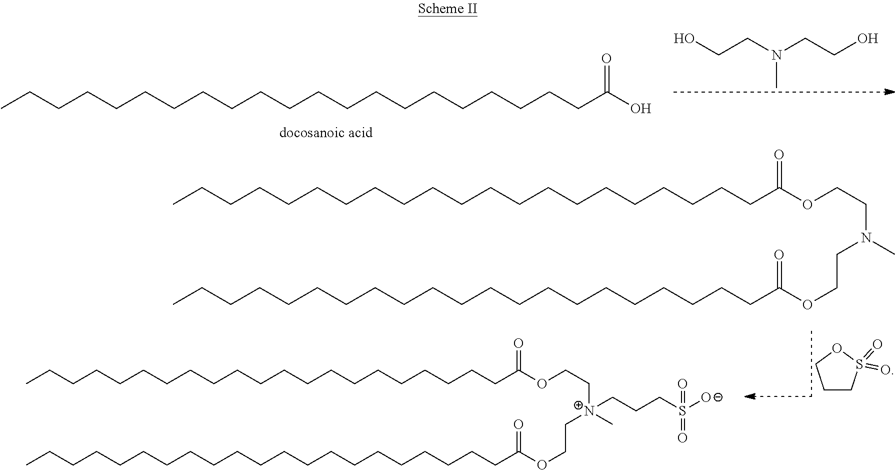

In one embodiment, a zwitterionic sulfobetaine-containing lipid can be obtained as set forth below in scheme III:

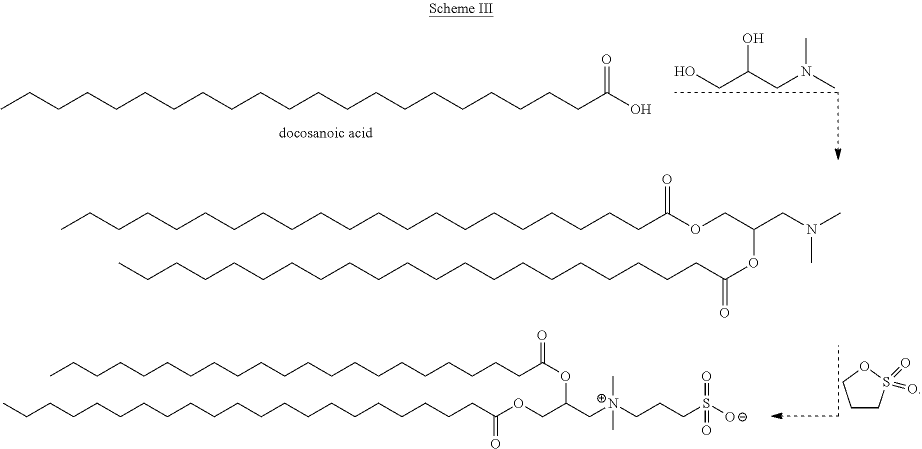

1. Ophthalmic Device with Entrapped Zwitterionic Sulfobetaine-Containing Lipid

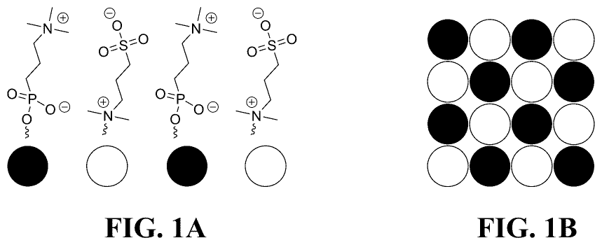

In accordance with a non-limiting illustrative embodiment, the zwitterionic sulfobetaine-containing lipids disclosed herein can be entrapped within an ophthalmic device thereby allowing the zwitterionic sulfobetaine-containing lipids to be released from the ophthalmic device during use. For example, a contact lens having the zwitterionic sulfobetaine-containing lipid disclosed entrapped within the lens will release the zwitterionic sulfobetaine-containing lipid upon contact with the tear film containing a lipid layer and an aqueous layer. The released zwitterionic sulfobetaine-containing lipid can then migrate to the polar lipid region (i.e., lipid layer) of the tear film, where it stabilizes the interface between the aqueous layer and the lipid layer of the tear film layers. It is believed that the zwitterionic sulfobetaine-containing lipid can form a two-dimensional alternating coulombically associated network with endogenous and/or exogenous zwitterionic phospholipids present in the tear film as illustrated in FIGS. 1A and 1B. Such coulombically associated networks would be expected to have lower average molecular area compared to either a zwitterionic sulfobetaine-containing lipid alone or a phospholipid alone.

The zwitterionic sulfobetaine-containing lipids can be entrapped within an ophthalmic device by methods known in the art. For example, in some embodiments, a zwitterionic sulfobetaine-containing lipid disclosed herein can be entrapped in an ophthalmic device by:
subjecting an ophthalmic device-forming mixture comprising one or more ophthalmic device-forming monomers and a zwitterionic sulfobetaine-containing lipid comprising a zwitterionic sulfobetaine group attached to one or more lipid groups each comprising at least one fatty acid acyl chain to polymerization conditions to provide a polymerized ophthalmic device, and
hydrating the polymerized ophthalmic device.

In some embodiments, the zwitterionic sulfobetaine-containing lipids disclosed herein can be present in the ophthalmic device-forming monomeric mixture in an amount ranging from about 0.1 wt. % to about 5.0 wt. %, based on the total weight of the ophthalmic device-forming monomeric mixture. In another illustrative embodiment, the zwitterionic sulfobetaine-containing lipids disclosed herein can be present in the ophthalmic device-forming monomeric mixture in an amount ranging from about 0.25 wt. % to about 2.0 wt. %, based on the total weight of the ophthalmic device-forming monomeric mixture.

In some embodiments, an ophthalmic device-forming monomeric mixture can include one or more hydrophilic monomers. Suitable one or more hydrophilic monomers include, for example, unsaturated carboxylic acids, acrylamides, vinyl lactams, poly(alkyleneoxy)(meth)acrylates, hydroxyl-containing-(meth)acrylates, hydrophilic vinyl carbonates, hydrophilic vinyl carbamates, hydrophilic oxazolones, and poly(alkene glycols) functionalized with polymerizable groups and the like and mixtures thereof.

Representative examples of unsaturated carboxylic acids include methacrylic acid, acrylic acid and the like and mixtures thereof. Representative examples of amides include alkylamides such as N,N-dimethylacrylamide, N,N-dimethylmethacrylamide and the like and mixtures thereof. Representative examples of cyclic lactams include N-vinyl-2-pyrrolidone, N-vinyl caprolactam, N-vinyl-2-piperidone and the like and mixtures thereof. Representative examples of hydroxyl-containing (meth)acrylates include 2-hydroxyethyl methacrylate, glycerol methacrylate and the like and mixtures thereof. Representative examples of functionalized poly(alkene glycols) include poly(diethylene glycols) of varying chain length containing monomethacrylate or dimethacrylate end caps. In one embodiment, the poly(alkene glycol) polymer contains at least two alkene glycol monomeric units. Still further examples are the hydrophilic vinyl carbonate or vinyl carbamate monomers disclosed in U.S. Pat. No. 5,070,215, and the hydrophilic oxazolone monomers disclosed in U.S. Pat. No. 4,910,277. Other suitable hydrophilic monomers will be apparent to one skilled in the art. Mixtures of the foregoing hydrophilic monomers can also be used in the ophthalmic device-forming monomeric mixtures herein.

In some embodiments, the one or more hydrophilic monomers can be present in the ophthalmic device-forming monomeric mixture in an amount ranging from about 30 wt. % to about 90 wt. %, based on the total weight of the ophthalmic device-forming monomeric mixture. In another illustrative embodiment, the one or more hydrophilic monomers can be present in the ophthalmic device-forming monomeric mixture in an amount ranging from about 45 wt. % to about 75 wt. %, based on the total weight of the ophthalmic device-forming monomeric mixture.

In some embodiments, the ophthalmic device-forming monomeric mixture can further include one or more hydrophobic monomers. Suitable hydrophobic monomers include ethylenically unsaturated hydrophobic monomers such as, for example, (meth)acrylate-containing hydrophobic monomers, N-alkyl (meth)acrylamide-containing hydrophobic monomers, alkyl vinylcarbonate-containing hydrophobic monomers, alkyl vinylcarbamate-containing hydrophobic monomers, fluoroalkyl (meth)acrylate-containing hydrophobic monomers, N-fluoroalkyl (meth)acrylamide-containing hydrophobic monomers, N-fluoroalkyl vinylcarbonate-containing hydrophobic monomers, N-fluoroalkyl vinylcarbamate-containing hydrophobic monomers, silicone-containing (meth)acrylate-containing hydrophobic monomers, (meth)acrylamide-containing hydrophobic monomers, vinyl carbonate-containing hydrophobic monomers, vinyl carbamate-containing hydrophobic monomers, styrenic-containing hydrophobic monomers, polyoxypropylene (meth)acrylate-containing hydrophobic monomers and the like and mixtures thereof.

In a non-limiting illustrative embodiment, the one or more hydrophobic monomers can be represented by the structure of Formula I:

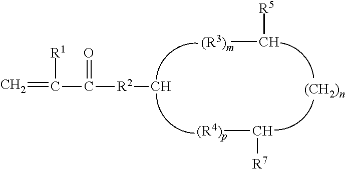

wherein R¹ is methyl or hydrogen; R² is —O— or —NH—; R³ and R⁴ are independently a divalent radical selected from the group consisting of —CH₂—, —CHOH— and —CHR⁶—; R⁵ and R⁶ are independently a branched C₃-C₈ alkyl group; R⁷ is hydrogen or —OH; n is an integer of at least 1, and m and p are independently 0 or an integer of at least 1, provided that the sum of m, p and n is 2, 3, 4 or 5.

Representative examples of one or more hydrophobic monomers represented by the structure of Formula I include, but are not limited to, 4-t-butyl-2-hydroxycyclohexyl methacrylate (TBE); 4-t-butyl-2-hydroxycyclopentyl methacrylate; 4-t-butyl-2-hydroxycyclohexyl methacrylamide (TBA); 6-isopentyl-3-hydroxycyclohexyl methacrylate; 2-isohexyl-5-hydroxycyclopentyl methacrylamide, 4-t-butylcyclohexyl methacrylate, isobornyl methacrylate, adamantyl methacrylate, n-butyl methacrylate, n-hexyl methacrylate, lauryl methacrylate, benzyl methacrylate, and the like. In one embodiment, one or more hydrophobic monomers include compounds of Formula I wherein R³ is —CH₂—, m is 1 or 2, p is 0, and the sum of m and n is 3 or 4.

In some embodiments, the one or more hydrophobic monomers can be present in the ophthalmic device-forming monomeric mixture in an amount ranging from about 0.5 wt. % to about 25 wt. %, based on the total weight of the ophthalmic device-forming monomeric mixture.

In some embodiments, the ophthalmic device-forming monomeric mixture can further include one or more crosslinking agents. Suitable crosslinking agents for use herein are known in the art. In illustrative embodiments, the one or more crosslinking agents are bi- or polyfunctional crosslinking agents comprising two or more reactive functional groups. In an embodiment, the one or more crosslinking agents have at least two polymerizable functional groups. Representative examples of crosslinking agents include divinylbenzene, allyl methacrylate, ethyleneglycol dimethacrylate, tetraethyleneglycol dimethacrylate, 1,4-butanediol diglycidyl ether, polyethyleneglycol dimethacrylate, vinyl carbonate derivatives of the glycol dimethacrylates, and methacryloxyethyl vinylcarbonate. However, other crosslinking agents are contemplated and the foregoing list is merely exemplary.

In some embodiments, the one or more crosslinking agents are used in the ophthalmic device-forming monomeric mixture in amounts of less than about 5 wt. %, and generally less than about 2 wt. %, e.g., from about 0.1 wt. % to about 5 wt. %, or from about 0.1 wt. % to about 2 wt. %, based on the total weight of the ophthalmic device-forming monomeric mixture.

It is particularly useful to employ biocompatible materials herein including both soft and rigid materials commonly used for contact lenses. In general, non-hydrogel materials are hydrophobic polymeric materials that do not contain water in their equilibrium state. Typical non-hydrogel materials comprise silicone acrylics, such as those formed bulky silicone monomer (e.g., tris(trimethylsiloxy)silylpropyl methacrylate, commonly known as “TRIS” monomer), methacrylate end-capped poly(dimethylsiloxane) prepolymer, or silicones having fluoroalkyl side groups (polysiloxanes are also commonly known as silicone polymers).

On the other hand, hydrogel materials comprise hydrated, cross-linked polymeric systems containing water in an equilibrium state. Hydrogel materials contain about 5 wt. % water or more (up to, for example, about 80 wt. %). In one embodiment, hydrogel materials, include silicone hydrogel materials. In another embodiment, hydrogel materials include vinyl functionalized polydimethylsiloxanes copolymerized with hydrophilic monomers as well as fluorinated methacrylates and methacrylate functionalized fluorinated polyethylene oxides copolymerized with hydrophilic monomers. Representative examples of suitable hydrogel materials for use herein include those disclosed in U.S. Pat. Nos. 5,310,779; 5,387,662; 5,449,729; 5,512,205; 5,610,252; 5,616,757; 5,708,094; 5,710,302; 5,714,557 and 5,908,906, the contents of which are incorporated by reference herein.

A wide variety of materials can be used herein, and silicone hydrogel contact lens materials are particularly preferred. Silicone hydrogels generally have a water content greater than about 5 wt. % and more commonly between about 10 wt. % to about 80 wt. %. Such materials are usually prepared by polymerizing a mixture containing at least one silicone-containing monomer and at least one hydrophilic monomer. Typically, either the silicone-containing monomer or the hydrophilic monomer functions as a crosslinking agent (a crosslinker being defined as a monomer having multiple polymerizable functionalities) or a separate crosslinker may be employed. Applicable silicone-containing monomers for use in the formation of silicone hydrogels are well known in the art and numerous examples are provided in U.S. Pat. Nos. 4,136,250; 4,153,641; 4,740,533; 5,034,461; 5,070,215; 5,260,000; 5,310,779; and 5,358,995.

In some embodiments, the ophthalmic device-forming monomeric mixture can further include one or more silicon-containing monomers. Representative examples of applicable silicon-containing monomers include bulky siloxane monomers containing an ethylenically unsaturated reactive end group. The term “bulky” refers to groups of the siloxane monomers that are sterically and/or electronically encumbering, i.e., sterically hindering. In a non-limiting illustrative embodiment, suitable bulky siloxane monomers include, for example, a bulky polysiloxanylalkyl (meth)acrylic monomer, a bulky polysiloxanylalkyl carbamate monomer and mixtures thereof. A representative example of a bulky siloxane monomer can include a bulky siloxane monomer represented by a structure of Formula IIa:

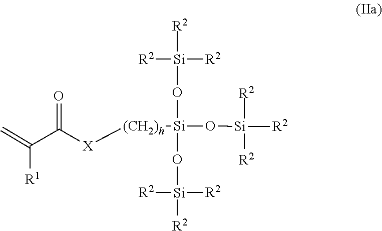

wherein X denotes —O— or —NR³—, where each R³ is hydrogen or a C₁-C₄ alkyl group; R¹ independently denotes hydrogen or methyl; each R² independently denotes a lower alkyl radical such as a C₁-C₆ group, a phenyl radical or a group represented by the following structure:

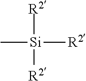

wherein each R^{2′} independently denotes a lower alkyl radical such as a C₁-C₆ group or a phenyl radical; and h is 1 to 10; or a bulky siloxane monomer represented by a structure of Formula IIb:

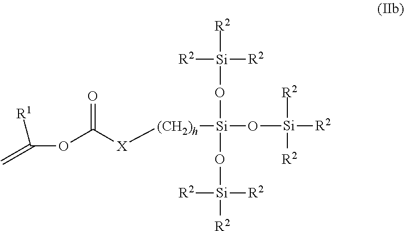

wherein X denotes —NR³— wherein R³ denotes hydrogen or a C₁-C₄ alkyl; R¹ denotes hydrogen or methyl; each R^{2′} independently denotes a lower alkyl radical such as a C₁-C₆ group, a phenyl radical or a group represented by the following structure:

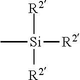

wherein each R^{2′} independently denotes a lower alkyl radical such as a C₁-C₆ group or a phenyl radical; and h is 1 to 10.

Representative examples of bulky siloxane monomers include 3-methacryloyloxypropyltris(trimethylsiloxy)silane or tris(trimethylsiloxy)silylpropyl methacrylate, sometimes referred to as TRIS, tris(trimethylsiloxy)silylpropyl vinyl carbamate, sometimes referred to as TRIS-VC, pentamethyldisiloxanyl methylmethacrylate, phenyltetramethyl-disiloxanylethyl acetate, and methyldi(trimethylsiloxy)methacryloxymethyl silane, (3-methacryloxy-2-hydroxy propoxy)propyl bis(trimethyl siloxy)methyl silane, sometimes referred to as Sigma and the like and mixtures thereof. In one embodiment, the bulky silicone-containing monomer is a tris(trialkylsiloxy)silylalkyl methacrylate-containing monomer such as a tris(trimethylsiloxy)silylpropyl methacrylate-containing monomer.

Such bulky monomers may be copolymerized with a silicone macromonomer, which is a poly(organosiloxane) capped with an unsaturated group at two or more ends of the molecule. U.S. Pat. No. 4,153,641 discloses, for example, various unsaturated groups such as acryloxy or methacryloxy groups.

Another class of representative silicone-containing monomers includes, but is not limited to, silicone-containing vinyl carbonate or vinyl carbamate monomers such as, for example, 1,3-bis[4-vinyloxycarbonyloxy)but-1-yl]tetramethyl-disiloxane; 3-(trimethylsilyl)propyl vinyl carbonate; 3-(vinyloxycarbonylthio)propyl-[tris(trimethylsiloxy)silane]; 3-[tris(trimethylsiloxy)silyl]propyl vinyl carbamate; 3-[tris(trimethylsiloxy)silyl]propyl allyl carbamate; 3-[tris(trimethylsiloxy)silyl]propyl vinyl carbonate; t-butyldimethylsiloxyethyl vinyl carbonate; trimethylsilylethyl vinyl carbonate; trimethylsilylmethyl vinyl carbonate and the like and mixtures thereof.

Another class of silicon-containing monomers includes polyurethane-polysiloxane macromonomers (also sometimes referred to as prepolymers), which may have hard-soft-hard blocks like traditional urethane elastomers. They may be end-capped with a hydrophilic monomer such as HEMA. Examples of such silicone urethanes are disclosed in a variety or publications, including Lai, Yu-Chin, “The Role of Bulky Polysiloxanylalkyl Methacryates in Polyurethane-Polysiloxane Hydrogels,” Journal of Applied Polymer Science, Vol. 60, 1193-1199 (1996). PCT Published Application No. WO 96/31792 discloses examples of such monomers, which disclosure is hereby incorporated by reference in its entirety. Further examples of silicone urethane monomers are represented by Formulae III and IV:

E(*D*A*D*G)ₐ*D*A*D*E′; or (III)

E(*D*G*D*A)ₐ*D*A*D*E′; or (IV)

wherein:
D independently denotes an alkyl diradical, an alkyl cycloalkyl diradical, a cycloalkyl diradical, an aryl diradical or an alkylaryl diradical having 6 to about 30 carbon atoms;
G independently denotes an alkyl diradical, a cycloalkyl diradical, an alkyl cycloalkyl diradical, an aryl diradical or an alkylaryl diradical having 1 to about 40 carbon atoms and which may contain ether, thio or amine linkages in the main chain;
* denotes a urethane or ureido linkage;
a is at least 1;
A independently denotes a divalent polymeric radical of Formula V:

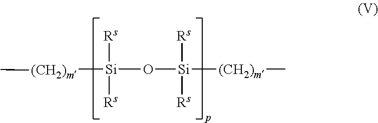

wherein each R^{s} independently denotes an alkyl or fluoro-substituted alkyl group having 1 to about 10 carbon atoms which may contain ether linkages between the carbon atoms; m′ is at least 1; and p is a number that provides a moiety weight of about 400 to about 10,000;
each of E and E′ independently denotes a polymerizable unsaturated organic radical represented by Formula VI:

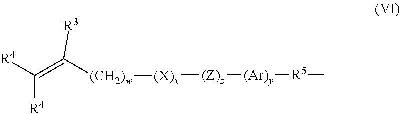

wherein: R³ is hydrogen or methyl;
R⁴ is hydrogen, an alkyl radical having 1 to 6 carbon atoms, or a —CO—Y—R⁶ radical wherein Y is —O—, —S— or —NH—;
R⁵ is a divalent alkylene radical having 1 to about 10 carbon atoms;
R⁶ is an alkyl radical having 1 to about 12 carbon atoms;
X denotes —CO— or —OCO—;
Z denotes —O— or —NH—;
Ar denotes an aromatic radical having about 6 to about 30 carbon atoms;
w is 0 to 6; x is 0 or 1; y is 0 or 1; and z is 0 or 1.

A preferred silicone-containing urethane monomer is represented by Formula VII:

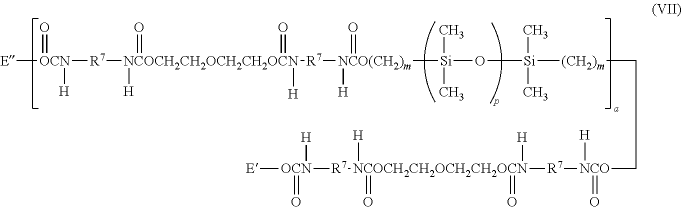

wherein m is at least 1 and is preferably 3 or 4, a is at least 1 and preferably is 1, p is a number which provides a moiety weight of about 400 to about 10,000 and is preferably at least about 30, R⁷ is a diradical of a diisocyanate after removal of the isocyanate group, such as the diradical of isophorone diisocyanate, and each E″ is a group represented by:

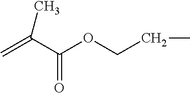

In another illustrative embodiment, a silicone hydrogel material comprises (in bulk, that is, in the ophthalmic device-forming monomeric mixture that is copolymerized) about 5 to about 50 percent, and preferably about 10 to about 25, by weight of one or more silicone macromonomers, about 5 to about 75 percent, and preferably about 30 to about 60 percent, by weight of one or more polysiloxanylalkyl (meth)acrylic monomers, and about 10 to about 50 percent, and preferably about 20 to about 40 percent, by weight of a hydrophilic monomer. In general, the silicone macromonomer is a poly(organosiloxane) capped with an unsaturated group at two or more ends of the molecule. In addition to the end groups in the above structural formulas, U.S. Pat. No. 4,153,641 discloses additional unsaturated groups, including acryloxy or methacryloxy. Fumarate-containing materials such as those disclosed in U.S. Pat. Nos. 5,310,779; 5,449,729 and 5,512,205 are also useful substrates in accordance with the illustrative embodiments. The silane macromonomer may be a silicon-containing vinyl carbonate or vinyl carbamate or a polyurethane-polysiloxane having one or more hard-soft-hard blocks and end-capped with a hydrophilic monomer.

Another class of representative silicone-containing monomers includes fluorinated monomers. Such monomers have been used in the formation of fluorosilicone hydrogels to reduce the accumulation of deposits on contact lenses made therefrom, as disclosed in, for example, U.S. Pat. Nos. 4,954,587; 5,010,141 and 5,079,319. Also, the use of silicone-containing monomers having certain fluorinated side groups, i.e., —(CF₂)—H, have been found to improve compatibility between the hydrophilic and silicone-containing monomeric units. See, e.g., U.S. Pat. Nos. 5,321,108 and 5,387,662.

The above silicone materials are merely exemplary, and other materials for use as substrates that have been disclosed in various publications and are being continuously developed for use in ophthalmic devices such as contact lenses can also be used.

In some embodiments, the ophthalmic device-forming monomeric mixture can further include a reactive (polymerizable) ultraviolet (UV) light absorber and/or a reactive blue-light absorber. Suitable reactive UV light absorbers can be any known reactive UV absorber. In non-limiting illustrative embodiments, suitable reactive UV light absorbers include, for example, 2-(2′-hydroxy-3′-methallyl-5′-methylphenyl)benzotriazole, commercially available as o-Methallyl Tinuvin P (“oMTP”) from Polysciences, Inc., Warrington, Pa., 3-(2H-benzo[d][1,2,3]triazol-2-yl)-4-hydroxyphenylethyl methacrylate, and 2-(3-(tert-butyl)-4-hydroxy-5-(5-methoxy-2H-benzo[d][1,2,3]triazol-2-yl)phenoxy)ethyl methacrylate.

In illustrative embodiments, the UV light absorbers can be present in the ophthalmic device-forming monomeric mixture in an amount ranging from about 0.1 wt. % to about 5 wt. %, based on the total weight of the ophthalmic device-forming monomeric mixture.

Many reactive blue-light absorbing compounds are known. Preferred reactive blue-light absorbing compounds are those described in U.S. Pat. Nos. 5,470,932; 8,207,244; and 8,329,775, the contents of which are hereby incorporated by reference. In one embodiment, a blue-light absorbing dye is N-2-[3-(2′-methylphenylazo)-4-hydroxyphenyl]ethyl methacrylamide. In illustrative embodiments, the blue-light absorbers can be present in the ophthalmic device-forming monomeric mixture in an amount ranging from about 0.005 wt. % to about 1 wt. %, based on the total weight of the ophthalmic device-forming monomeric mixture.

The ophthalmic device-forming monomeric mixtures disclosed herein may further contain, as necessary and within limits not to impair the purpose and effect of the illustrative embodiments disclosed herein, various additives such as an antioxidant, coloring agent, toughening agents and the like and other constituents as is well known in the art.

The ophthalmic device discussed above can be prepared by polymerizing the ophthalmic device-forming monomeric mixture containing the one or more ophthalmic device-forming monomers and a zwitterionic sulfobetaine-containing lipid disclosed herein to form a polymerization product that can be subsequently formed into the appropriate shape by, for example, lathing, injection molding, compression molding, cutting and the like. For example, in producing contact lenses, the initial ophthalmic device-forming monomeric mixture may be polymerized in tubes to provide rod-shaped articles, which are then cut into buttons. The buttons may then be lathed into contact lenses.

Alternately, the ophthalmic device such as a contact lens may be cast directly in molds, e.g., polypropylene molds, from the mixtures, e.g., by spincasting and static casting methods. Spincasting methods are disclosed in U.S. Pat. Nos. 3,408,429 and 3,660,545, and static casting methods are disclosed in U.S. Pat. Nos. 4,113,224, 4,197,266, and 5,271,875. Spincasting methods involve charging the mixtures to be polymerized to a mold, and spinning the mold in a controlled manner while exposing the mixture to a radiation source such as UV light. Static casting methods involve charging the ophthalmic device-forming monomeric mixture between two mold sections, one mold section shaped to form the anterior lens surface and the other mold section shaped to form the posterior lens surface, and curing the mixture while retained in the mold assembly to form a lens, for example, by free radical polymerization of the mixture.

Examples of free radical reaction techniques to cure the lens material include thermal radiation, infrared radiation, electron beam radiation, gamma radiation, ultraviolet (UV) radiation, and the like; or combinations of such techniques may be used. U.S. Pat. No. 5,271,875 describes a static cast molding method that permits molding of a finished lens in a mold cavity defined by a posterior mold and an anterior mold. As an additional method, U.S. Pat. No. 4,555,732 discloses a process where an excess of a mixture is cured by spincasting in a mold to form a shaped article having an anterior lens surface and a relatively large thickness, and the posterior surface of the cured spincast article is subsequently lathed to provide a contact lens having the desired thickness and posterior lens surface.

Polymerization may be facilitated by exposing the mixture to heat and/or radiation, such as ultraviolet light, visible light, or high energy radiation. A polymerization initiator may be included in the mixture to facilitate the polymerization step. Representative examples of free radical thermal polymerization initiators include organic peroxides such as acetyl peroxide, lauroyl peroxide, decanoyl peroxide, stearoyl peroxide, benzoyl peroxide, tertiarylbutyl peroxypivalate, peroxydicarbonate, and the like. Representative UV initiators are those known in the art and include benzoin methyl ether, benzoin ethyl ether, Darocure® 1173, 1164, 2273, 1116, 2959, 3331 (EM Industries) and Irgacure® 651, 184 and 2959 (Ciba-Geigy), 2,2′Azobis(2-methylpropionitrile) (VAZO 64) and the like. Generally, the initiator will be employed in the mixture at a concentration of about 0.01 to about 5 percent by weight of the total mixture.

Polymerization is generally performed in a reaction medium, such as, for example, a solution or dispersion using a solvent, e.g., water or an alkanol containing from 1 to 4 carbon atoms such as methanol, ethanol or propan-2-ol. Alternatively, a mixture of any of the above solvents may be used.

Generally, polymerization can be carried out for about 15 minutes to about 72 hours, and under an inert atmosphere of, for example, nitrogen or argon. If desired, the resulting polymerization product can be dried under vacuum, e.g., for about 5 to about 72 hours or left in an aqueous solution prior to use.

Polymerization of the ophthalmic device-forming monomeric mixtures will yield a polymer, that when hydrated, preferably forms a hydrogel. When producing a hydrogel lens, the ophthalmic device-forming monomeric mixture may further include at least a diluent that is ultimately replaced with water when the polymerization product is hydrated to form a hydrogel. The amount of diluent used should be less than about 50 wt. %, and in most cases, the diluent content will be less than about 30 wt. %. However, in a particular polymer system, the actual limit will be dictated by the solubility of the various monomers in the diluent. In order to produce an optically clear copolymer, it is important that a phase separation leading to visual opacity does not occur between the comonomers and the diluent, or the diluent and the final copolymer.

Furthermore, the maximum amount of diluent which may be used will depend on the amount of swelling the diluent causes the final polymers. Excessive swelling will or may cause the copolymer to collapse when the diluent is replaced with water upon hydration. Suitable diluents include, but are not limited to, ethylene glycol, glycerine, liquid poly(ethylene glycol), alcohols, alcohol/water mixtures, ethylene oxide/propylene oxide block copolymers, low molecular weight linear poly(2-hydroxyethyl methacrylate), glycol esters of lactic acid, formamides, ketones, dialkylsulfoxides, butyl carbitol, and the like and mixtures thereof.

If necessary, it may be desirable to remove residual diluent from the lens before edge-finishing operations which can be accomplished by evaporation at or near ambient pressure or under vacuum. An elevated temperature can be employed to shorten the time necessary to evaporate the diluent. The time, temperature and pressure conditions for the solvent removal step will vary depending on such factors as the volatility of the diluent and the specific monomeric components, as can be readily determined by one skilled in the art.

In accordance with another non-limiting illustrative embodiment, an ophthalmic device having a zwitterionic sulfobetaine-containing lipid entrapped in the ophthalmic device can be obtained by:
soaking an ophthalmic device in one or more first solvent solutions to swell the ophthalmic device, thereby providing a swelled ophthalmic device,
soaking the swelled ophthalmic device in one or more second solvent solutions to de-swell the ophthalmic device, thereby providing a de-swelled ophthalmic device, and
sterilizing the de-swelled ophthalmic device,
wherein the one or more first solvent solutions, the one or more second solvent solutions or both comprise the zwitterionic sulfobetaine-containing lipid comprising a zwitterionic sulfobetaine group attached to one or more lipid groups each comprising at least one fatty acid acyl chain.

In step (a) of the illustrative process, the ophthalmic device is soaked in one or more first solvent solutions for a time period sufficient to swell the ophthalmic device, thereby providing a swelled ophthalmic device. In general, the one or more first solvent solutions include a solvent capable of swelling the ophthalmic lens. In one embodiment, the one or more first solvent solutions include, for example, a low molecular weight alcohol solvent, an aliphatic hydrocarbon solvent, a cycloaliphatic hydrocarbon solvent, a ketone solvent, a nitrile solvent, an ether solvent, an amido group-containing solvent and mixtures thereof. Suitable low molecular weight alcohols include, for example, low molecular weight alcohols having about 1 to about 13 carbon atoms and/or a molecular weight of no greater than about 200. A suitable low molecular alcohol can be selected from a variety of low-molecular-weight monohydric alcohols, each comprising about 1 to about 13 carbon atoms. Suitable monohydric alcohols include, for example, methanol, ethanol, propanol, isopropyl alcohol, butanol, isobutyl alcohol, tert-butyl alcohol, hexanol, 2-ethylhexanol, dodecanol, and the like. Suitable aliphatic or cycloaliphatic hydrocarbon solvents include, for example, pentane, hexane, heptane, cyclohexane and the like.

Suitable ketone solvents include, for example, acetone, methyl ethyl ketone, diethyl ketone, methyl propyl ketone, methyl isopropyl ketone, ethyl propyl ketone, ethyl isopropyl ketone, dipropyl ketone, diisopropyl ketone, methyl butyl ketone, methyl isobutyl ketone, methyl sec butyl ketone, methyl tert-butyl ketone, ethyl butyl ketone, ethyl isobutyl ketone, ethyl sec-butyl ketone, ethyl tert-butyl ketone, propyl butyl ketone, isopropyl butyl ketone, propyl isobutyl ketone, propyl sec-butyl ketone, propyl tert butyl ketone, isopropyl isobutyl ketone, isopropyl sec-butyl ketone, isopropyl tert-butyl ketone, dibutyl ketone, diisobutyl ketone, di-sec-butyl ketone, di-tert-butyl ketone, butyl isobutyl ketone, butyl sec-butyl ketone, butyl tert-butyl ketone, isobutyl sec-butyl ketone, isobutyl tert-butyl ketone, sec-butyl tert-butyl ketone, 5-heptanone, 5-methyl-2-hexanone (methyl isoamyl ketone), 4-methyl-2-hexanone, 3-methyl-2-hexanone, 3,4-dimethyl-2-pentanone, 3,3-dimethyl-2-pentanone, 4,4-dimethyl-2-pentanone, 3-octanone, 4-methyl-3-heptanone, 5-methyl-3-heptanone, 6-methyl-3-heptanone, 4,4-dimethyl-3-hexanone, 4,5-dimethyl-3-hexanone, 5,5-dimethyl-3-hexanone, 4-nonanone, 5-methyl-4-octanone, 6-methyl-4-octanone, 7-methyl-4-octanone, 5,5-dimethyl-4-neptanone, 5,6-dimethyl-4-heptanone, 6,6-dimethyl-4-heptanone, 2-undecanone, cyclopropanone, cyclobutanone, cyclopentanone, cyclohexanone, cycloheptanone, cyclooctanone, cyclononanone, cyclodecanone, cycloundecanone, cyclododecanone and the like and combinations thereof. In one embodiment, a ketone solvent is acetone.

Suitable nitrile solvents include, for example, saturated or unsaturated aliphatic, alicyclic, or aromatic compounds containing a nitrile group. Included within the nitriles are compounds containing heteroatom such as those selected from Groups 13, 14, 15, 16 and 17 of the Periodic Table of Elements. Representative examples of nitriles for use herein include acetonitrile; propionitrile; isopropionitrile; butyronitrile; isobutyronitrile; valeronitrile; isovaleronitrile; trimethylacetonitrile; hexanenitrile; heptanenitrile; heptyl cyanide; octyl cyanide; undecanenitrile; malononitrile; succinonitrile; glutaronitrile; adiponitrile; sebaconitrile; allyl cyanide; acrylonitrile; crotononitrile; methacrylonitrile; fumaronitrile; tetracyanoethylene; cyclopentanecarbonitrile; cyclohexanecarbonitrile; dichloroacetonitrile; fluoroacetonitrile; trichloroacetonitrile; benzonitrile; benzyl cyanide; 2-methylbenzyl cyanide; 2-chlorobenzonitrile; 3-chlorobenzonitrile; 4-chlorobenzonitrile; o-tolunitrile; m-tolunitrile; p-tolunitrile and the like and mixtures thereof. In one embodiment, a nitrile solvent is acetonitrile.

Suitable ether solvents include, for example, dialkyl ethers wherein the alkyl groups are the same or different and are from 1 to about 12 carbon atoms. Representative examples of an ether solvent include dimethylether, diethylether, di-i-propylether; dioxane, tetrahydrofuran, pyran and the like and mixtures thereof. In one embodiment, an ether solvent is tetrahydrofuran.

Suitable amido group-containing solvents include, for example, dimethyl formamide, N-methyl formanilide, N-formyl piperidine, N-formyl morpholine, dimethyl acetamide, N-methyl pyrrolidone, N,N-dimethyl benzamide and mixtures thereof. In one embodiment, an amido group-containing solvent is N-methyl pyrrolidone.

In one embodiment, the one or more first solvent solutions can further include water in combination with any of the foregoing first solvents. For example, the one or more first solvent solutions can be a blend containing from about 25 wt. % to about 75 wt. % one or more first solvent solutions and from about 75 wt. % to about 25 wt. % water. In another embodiment, a blend can contain from about 40 wt. % to about 60 wt. % one or more first solvent solutions and from about 60 wt. % to about 40 wt. % water.

The ophthalmic device is soaked in the one or more first solvent solutions for a time period sufficient to swell the ophthalmic device, thereby providing a swelled ophthalmic device. In general, the ophthalmic device is soaked in the one or more first solvent solutions for a time period ranging from about 5 minutes to about 120 minutes. In one embodiment, the ophthalmic device is soaked in the one or more first solvent solutions for a time period ranging from about 5 minutes to about 60 minutes. In one embodiment, the ophthalmic device is soaked in the one or more first solvent solutions for a time period ranging from about 10 minutes to about 35 minutes.

In one embodiment, the ophthalmic device is soaked in a series of the one or more first solvent solutions. For example, the ophthalmic device is first soaked in the one or more first solvent solutions or blend of the one or more first solvents and water as discussed hereinabove for a time period ranging from about 5 minutes to about 30 minutes, or from about 5 minutes to about 20 minutes. Next, the ophthalmic device is removed from the solution or blend and soaked in another solvent solution of any of the foregoing one or more first solvents for a time period ranging from about 5 minutes to about 120 minutes. In one embodiment, the ophthalmic device is soaked in another solvent solution for a time period ranging from about 5 minutes to about 60 minutes. In one embodiment, the ophthalmic device is soaked in another solvent solution for a time period ranging from about 10 minutes to about 35 minutes.

In some embodiments, the one or more first solvent solutions include a zwitterionic sulfobetaine-containing lipid disclosed herein to entrap the zwitterionic sulfobetaine-containing lipid in the ophthalmic device when swelling the ophthalmic device. In some embodiments, the one or more first solvent solutions include a zwitterionic sulfobetaine-containing lipid disclosed herein in an amount of about 0.1 wt. % to about 10 wt. %, based on the total weight of the one or more first solvent solutions.

In step (b) of the illustrative process, the swelled ophthalmic device is soaked in one or more second solvent solutions to N, N de-swell the ophthalmic device, thereby providing a de-swelled ophthalmic device. Suitable second solvent solutions include, for example, water and any of the foregoing low molecular weight alcohol solvent, an aliphatic hydrocarbon solvent, a cycloaliphatic hydrocarbon solvent, a ketone solvent, a nitrile solvent, an ether solvent, or an amido group-containing solvents discussed hereinabove.

In one embodiment, the one or more second solvent solutions include a blend of water together with any of the low molecular weight alcohol solvents, aliphatic hydrocarbon solvents, cycloaliphatic hydrocarbon solvents, ketone solvents, nitrile solvents, ether solvents, and amido group-containing solvent. For example, the one or more second solvent solutions can be a blend containing from about 25 wt. % to about 75 wt. % one or more second solvent solutions such as a low molecular weight alcohol and from about 75 wt. % to about 25 wt. % water. In another embodiment, a blend can contain from about 40 wt. % to about 60 wt. % one or more second solvent solutions such as a low molecular weight alcohol and from about 60 wt. % to about 40 wt. % water.

In some embodiments, the one or more second solvent solutions include a zwitterionic sulfobetaine-containing lipid disclosed herein. In the case where the one or more first solvent solutions include the zwitterionic sulfobetaine-containing lipid disclosed herein, it may be advantageous to further include the zwitterionic sulfobetaine-containing lipid disclosed herein in the one or more second solvent solutions to ensure convective mass transfer into the ophthalmic device during swelling while limiting convective mass transfer out of the ophthalmic device during de-swelling to N, N de-swell the ophthalmic device, thereby providing a de-swelled ophthalmic device and entrap the zwitterionic sulfobetaine-containing lipid in the de-swelled ophthalmic device. In some embodiments, the one or more second solvent solutions include a zwitterionic sulfobetaine-containing lipid disclosed herein in an amount of about 0.1 wt. % to about 10 wt. %, based on the total weight of the one or more second solvent solutions.

The ophthalmic device is soaked in the one or more second solvent solutions for a time period sufficient to de-swell the ophthalmic device and entrap the zwitterionic sulfobetaine-containing lipid. In general, the ophthalmic device is soaked in the one or more second solvent solutions for a time period ranging from about 5 minutes to about 120 minutes. In one embodiment, the ophthalmic device is soaked in the one or more second solvent solutions for a time period ranging from about 10 minutes to about 35 minutes.

In one embodiment, the ophthalmic device is soaked in a series of the one or more second solvent solutions. For example, the ophthalmic device is first soaked in the one or more second solvent solutions comprising the low molecular weight alcohol solvent and the zwitterionic sulfobetaine-containing lipid for a time period ranging from about 5 minutes to about 20 minutes. Next, the ophthalmic device is removed from the solvent solution and further soaked in another solvent solution comprising a blend of the same or different second low molecular weight alcohol solvent and water and the zwitterionic sulfobetaine-containing lipid for a time period ranging from about 5 minutes to about 20 minutes.

After the ophthalmic device has been de-swelled, the de-swelled ophthalmic device is removed and optionally soaked in one more series of water solutions to further de-swell the device. In general, the de-swelled ophthalmic device is soaked in the one or more water solutions for a time period ranging from about 5 minutes to about 20 minutes.

In step (c), the de-swelled ophthalmic device is sterilized. In one embodiment, the de-swelled ophthalmic device is sterilized by submerging the de-swelled ophthalmic device in a borate buffered saline and then subjecting it to autoclave conditions for at least about 5 minutes, or at least about 20 minutes or at least 24 hours or up to about 72 hours. The sterilized de-swelled ophthalmic device is then rinsed with water and positioned in their packaging with borate buffered saline. The package is sealed and again the ophthalmic device is subjected to autoclave conditions.

Alternatively, the de-swelled ophthalmic device can be placed in a container that includes a receptacle portion to hold the de-swelled ophthalmic device and a sterile packaging solution. Examples of the container are conventional ophthalmic device blister packages. This receptacle, containing the de-swelled ophthalmic device immersed in the packaging solution, is hermetically sealed, for example, by sealing lidstock on the package over the receptacle. For example, the lidstock is sealed around a perimeter of the receptacle. The packaging solution and the de-swelled ophthalmic device are sterilized while sealed in the package receptacle. Examples of sterilization techniques include subjecting the solution and the de-swelled ophthalmic device to thermal energy, microwave radiation, gamma radiation or ultraviolet radiation. A specific example involves heating the packaging solution and the de-swelled ophthalmic device, while sealed in the package container, to a temperature of at least 100 °C, or at least 120 °C, such as by autoclaving.

### 2. Packaging Systems

In a non-limiting illustrative embodiment, an unused ophthalmic device as described above can be transferred to an individual lens package containing a buffered saline solution including the zwitterionic sulfobetaine-containing lipid disclosed herein. Generally, a packaging system for the storage of an unused ophthalmic device includes at least a sealed container containing an unused ophthalmic device immersed in an aqueous packaging solution containing the zwitterionic sulfobetaine-containing lipid disclosed herein. In one embodiment, the sealed container is a hermetically sealed blister-pack, in which a concave well containing an ophthalmic device such as a contact lens is covered by a metal or plastic sheet adapted for peeling in order to open the blister-pack. The sealed container may be any suitable generally inert packaging material providing a reasonable degree of protection to the lens, preferably a plastic material such as polyalkylene, PVC, polyamide, and the like.

In some embodiments, the amount of the zwitterionic sulfobetaine-containing lipid disclosed herein employed in an aqueous packaging solution for storing an unused ophthalmic device in a packaging system is an amount effective to maintain the surface properties and internal zwitterionic material concentration of the ophthalmic device. It is believed that the zwitterionic sulfobetaine-containing lipid disclosed herein enhances initial and extended comfort when an unused ophthalmic device such as a contact lens, packaged in the aqueous packaging solution and then removed from the packaging system, is placed on the eye for wearing. In an illustrative embodiment, the concentration of the zwitterionic sulfobetaine-containing lipid disclosed herein in the aqueous packaging solution can range from about 0.01% w/w to about 20% w/w. In an illustrative embodiment, the concentration of the zwitterionic sulfobetaine-containing lipid disclosed herein in the aqueous packaging solution can range from about 0.02% w/w to about 2% w/w.

The aqueous packaging solutions are physiologically compatible. Specifically, the solution must be “ophthalmically safe” for use with a lens such as a contact lens, meaning that a contact lens treated with the solution is generally suitable and safe for direct placement on the eye without rinsing, that is, the solution is safe and comfortable for daily contact with the eye via a contact lens that has been wetted with the solution. An ophthalmically safe solution has a tonicity and pH that is compatible with the eye and includes materials, and amounts thereof, that are non-cytotoxic according to ISO standards and U.S. Food & Drug Administration (FDA) regulations.

The aqueous packaging solution should also be sterile in that the absence of microbial contaminants in the product prior to release must be statistically demonstrated to the degree necessary for such products. The liquid media useful herein is selected to have no substantial detrimental effect on the lens being treated or cared for and to allow or even facilitate the present lens treatment or treatments. In one embodiment, the liquid media is aqueous-based. A particularly useful aqueous liquid medium is that derived from saline, for example, a conventional saline solution or a conventional buffered saline solution.

The pH of the aqueous packaging solutions should be maintained within the range of about 6 to about 9, or from about 6.5 to about 7.8. Suitable buffers may be added, such as boric acid, sodium borate, potassium citrate, citric acid, sodium bicarbonate, TRIS and various mixed phosphate buffers (including combinations of Na₂HPO₄, NaH₂PO₄ and KH₂PO₄) and mixtures thereof. Generally, buffers will be used in amounts ranging from about 0.05 to about 2.5 percent by weight of the solution. In one embodiment, buffers will be used in amounts ranging from about 0.1 to about 1.5 percent by weight of the solution. In one embodiment, the packaging solutions of this invention will contain a borate buffer, e.g., a borate buffer containing one or more of boric acid, sodium borate, potassium tetraborate, potassium metaborate or mixtures thereof. In another embodiment, the packaging solutions of this invention will contain a phosphate buffer.

Typically, the aqueous packaging solutions are also adjusted with tonicity agents, to approximate the osmotic pressure of normal lacrimal fluids which is equivalent to a 0.9 percent solution of sodium chloride or 2.5 percent of glycerol solution. The aqueous packaging solutions are made substantially isotonic with physiological saline used alone or in combination, otherwise if simply blended with sterile water and made hypotonic or made hypertonic the lenses will lose their desirable optical parameters. Correspondingly, excess saline may result in the formation of a hypertonic solution which will cause stinging and eye irritation.

Suitable tonicity adjusting agents include, for example, sodium and potassium chloride, dextrose, glycerin, calcium and magnesium chloride and the like and mixtures thereof. These tonicity adjusting agents are typically used individually in amounts ranging from about 0.01% w/v to about 2.5% w/v. In one illustrative embodiment, the tonicity adjusting agents are used in amounts ranging from about 0.2% w/v to about 1.5% w/v. The tonicity agent will be employed in an amount to provide a final osmotic value of at least about 200 mOsmol/kg. In one illustrative embodiment, the tonicity adjusting agents are used in an amount to provide a final osmotic value of from about 200 mOsmol/kg to about 400 mOsmol/kg. In one illustrative embodiment, the tonicity adjusting agents are used in an amount to provide a final osmotic value of from about 250 mOsmol/kg to about 350 mOsmol/kg. In one illustrative embodiment, the tonicity adjusting agents are used in an amount to provide a final osmotic value of from about 280 mOsmol/kg to about 320 mOsmol/kg.

If desired, one or more additional components can be included in the aqueous packaging solution. Such additional component or components are chosen to impart or provide at least one beneficial or desired property to the packaging solution. In general, the additional components may be selected from components which are conventionally used in one or more ophthalmic device care compositions. Suitable additional components include, for example, cleaning agents, wetting agents, nutrient agents, sequestering agents, viscosity builders, contact lens conditioning agents, antioxidants, and the like and mixtures thereof. These additional components may each be included in the packaging solutions in an amount effective to impart or provide the beneficial or desired property to the packaging solutions. For example, such additional components may be included in the packaging solutions in amounts similar to the amounts of such components used in other, e.g., conventional, contact lens care products.

The method of packaging and storing an unused ophthalmic device such as an unused contact lens includes at least packaging an unused ophthalmic device immersed in the aqueous packaging solution described above. The method may include immersing the unused ophthalmic device in an aqueous packaging solution prior to delivery to the customer/wearer, directly following manufacture of the contact lens. Alternately, the packaging and storing in the aqueous packaging solution may occur at an intermediate point before delivery to the ultimate customer (wearer) but following manufacture and transportation of the lens in a dry state, wherein the dry lens is hydrated by immersing the lens in the packaging solution. Consequently, a package for delivery to a customer may include a sealed container containing one or more unused contact lenses immersed in an aqueous packaging solution according to the present invention.

In one illustrative embodiment, the steps leading to the present ophthalmic device packaging system includes (1) molding an ophthalmic device in a mold comprising at least a first and second mold portion, (2) hydrating and cleaning the ophthalmic device in a container comprising at least one of the mold portions, (3) introducing the aqueous packaging solution with the zwitterionic sulfobetaine-containing lipid disclosed herein into the container with the unused ophthalmic device supported therein, and (4) sealing the container. In one embodiment, the method also includes the step of sterilizing the contents of the container. Sterilization may take place prior to, or most conveniently after, sealing of the container and may be carried out by any suitable method known in the art, e.g., heat sterilized by autoclaving of the sealed container at temperatures of about 120 °C or higher, or sterilized by other known techniques such as UV, beta or gamma radiation or sterile filtration.

### 3. Aqueous Ophthalmic Solutions

In a non-limiting illustrative embodiment, the zwitterionic sulfobetaine-containing lipid disclosed herein can be in an aqueous ophthalmic solution. The aqueous ophthalmic solutions generally include compositions for direct instillation in the eye, including eye drop solutions and contact lens treating solutions distilled directly in the eye such as for rewetting a contact lens while worn as well as those that also qualify as a multi-purpose solution. Ophthalmic compositions also include compositions instilled indirectly in the eye, such as contact lens treating solutions for treating the contact lens prior to the lens being inserted on the eye or a packaging solution for storing the lens. The aqueous ophthalmic solution can be preservative-free aqueous ophthalmic solutions.

The aqueous ophthalmic solutions in accordance with this embodiment are physiologically compatible. Specifically, the aqueous ophthalmic solutions must be “ophthalmically safe” for use with a contact lens, meaning that a contact lens treated with the solution is generally suitable and safe for direct placement on the eye without rinsing, that is, the solution is safe and comfortable for daily contact with the eye via a contact lens that has been wetted with the solution. An ophthalmically safe composition has a tonicity and pH that is compatible with the eye and comprises materials, and amounts thereof, that are non-cytotoxic according to ISO (International Standards Organization) standards and U.S. FDA regulations. The compositions should be sterile in that the absence of microbial contaminants in the product prior to release must be statistically demonstrated to the degree necessary for such products.

In some embodiments, the zwitterionic sulfobetaine-containing lipid disclosed herein can be present in the aqueous ophthalmic solution in an amount ranging from about 0.001% w/w to about 10% w/w. In some embodiments, the zwitterionic sulfobetaine-containing lipid disclosed herein can be present in the aqueous ophthalmic solution in an amount ranging from about 0.1% w/w to about 2% w/w.

In some embodiments, the aqueous ophthalmic solutions may be in the form of drops and are useful as a component of a contact lens cleaning, disinfecting or conditioning composition containing such materials. In one embodiment, the compositions and/or solutions of the present invention may be formulated as a “multi-purpose solution”. A multi-purpose solution is useful for cleaning, disinfecting, storing, and rinsing a lens, particularly soft contact lenses. Multi-purpose solutions do not exclude the possibility that some wearers, for example, wearers particularly sensitive to chemical disinfectants or other chemical agents, may prefer to rinse or wet a contact lens with another solution, for example, a sterile saline solution prior to insertion of the lens. The term “multi-purpose solution” also does not exclude the possibility of periodic cleaners not used on a daily basis or supplemental cleaners for further removing proteins, for example, enzyme cleaners, which are typically used on a weekly basis. By the term “cleaning” is meant that the solution contains one or more agents in sufficient concentrations to loosen and remove loosely held lens deposits and other contaminants on the surface of a contact lens, which may be used in conjunction with digital manipulation (e.g., manual rubbing of the lens with a solution) or with an accessory device that agitates the solution in contact with the lens, for example, a mechanical cleaning aid.

Traditionally, multi-purpose solutions on the market have required a regimen involving mechanical rubbing of the lens with the multi-purpose solution, in order to provide the required disinfection and cleaning. Such a regimen is required under governmental regulatory authorities (e.g., the FDA or U.S. Food & Drug Administration (FDA)) for a Chemical Disinfection System that does not qualify as a Chemical Disinfecting Solution. In one embodiment of the present invention, it is possible to formulate a cleaning and disinfecting product that, on one hand, is able to provide improved cleaning and disinfection in the absence of a rubbing regimen and, on the other hand, is gentle enough to be used as a wetting agent, e.g. as an eye drop. For example, a product qualifying as a Chemical Disinfecting Solution must meet biocidal performance criteria established by the US FDA for Contact Lens Care Products (May 1, 1997) which criteria does not involve rubbing of the lenses. In one embodiment of the present invention, a composition is formulated to meet the requirements of the FDA or ISO Stand-Alone Procedure for contact lens disinfecting products. Similarly, the compositions of the present invention can be formulated to provide enhanced cleaning without the use of a rubbing regimen. Such formulations may ensure higher patient compliance and greater universal appeal than traditional multi-purpose disinfecting and cleaning products. A multi-purpose solution can have a viscosity of less than about 75 cps, or from about 1 to about 50 cps, or from about 1 to about 25 cps or at least about 95 percent weight by volume water in the total composition.

The aqueous ophthalmic solutions may contain, in addition to the zwitterionic sulfobetaine-containing lipid disclosed herein, one or more antimicrobial agents, preservatives and the like. The aqueous ophthalmic solutions generally include a primary antimicrobial agent. Antimicrobial agents suitable for use in the present invention include chemicals that derive their antimicrobial activity through a chemical or physiochemical interaction with the microbial organisms. These agents may be used alone or in combination.

Suitable known ophthalmically acceptable antimicrobial agents include, but are not limited to, a biguanide or a salt or free base thereof, quaternary ammonium compound or a salt thereof or free base thereof; terpene or derivative thereof, a branched, glycerol monoalkyl ether, a branched, glycerol monoalkyl amine, a branched, glycerol monoalkyl sulphide, a fatty acid monoester, wherein the fatty acid monoester comprises an aliphatic fatty acid portion having six to fourteen carbon atoms, and an aliphatic hydroxyl portion, amidoamine compound, and the like and combinations thereof.

Suitable biguanide antimicrobial agents for use in the ophthalmic compositions can be any biguanide or salt thereof known in the art. Representative biguanides include non-polymeric biguanides, polymeric biguanides, salts thereof, free bases thereof and the like and mixtures thereof. Representative non-polymeric biguanides are the bis(biguanides), such as alexidine, chlorhexidine, salts of alexidine, e.g., alexidine HCl, salts of chlorhexidine, alexidine free base, and the like and mixtures thereof. The salts of alexidine and chlorhexidine can be either organic or inorganic and are typically disinfecting nitrates, acetates, phosphates, sulfates, halides and the like.

Representative polymeric biguanides include polymeric hexamethylene biguanides (PHMB) (commercially available from Zeneca, Wilmington, Del.), their polymers and water-soluble salts. In one embodiment, water-soluble polymeric biguanides for use herein can have a number average molecular weight of at least about 1,000 or a number average molecular weight from about 1,000 to about 50,000. Suitable water-soluble salts of the free bases include, but are not limited to, hydrochloride, borate, acetate, gluconate, sulfonate, tartrate and citrate salts. Generally, the hexamethylene biguanide polymers, also referred to as polyaminopropyl biguanide (PAPB), have number average molecular weights of up to about 100,000. Such compounds are known and are disclosed in U.S. Pat. No. 4,758,595 which is incorporated herein by reference.

PHMB is best described as a polymeric biguanide composition comprising at least three and preferably at least six biguanide polymers, which we refer to as PHMB-A, PHMB-CG and PHMB-CGA, the general chemical structures of which are depicted below.

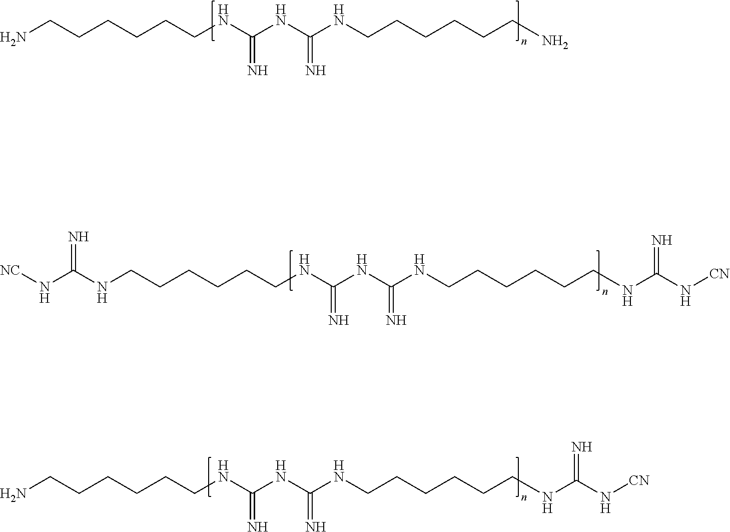

For each of these polymers, “n” represents the average number of repeating groups. Actually, a distribution of polymer length would exist for each of the polymers shown. The prior synthetic routes to PHMB provided a polymeric biguanide composition with about 50% by weight of the polymeric composition as PHMB-CGA, that is, having a cyanoguanidino end cap on one end and an amine on the other end, about 25% by weight PHMB-A and about 25% by weight PHMB-CG. Given this approximate weight ratio of the three major PHMB polymers above, the percentage of cyanoguardino end caps is also about 50% of the total number of terminal groups. In this application we refer to this conventional polymeric biguanide composition as poly(hexamethylene biguanide) or PHMB.

A polymeric biguanide composition comprising less than 18 mole % of terminal amine groups as measured by ¹³CNMR can also be used. The polymeric biguanide composition can also be characterized by a relative increase in the molar concentration of terminal guanidine groups or terminal cyanoguardino groups. For example, in one embodiment, the biguanide composition comprises less than about 18 mole % of terminal amine groups and about 40 mol % or greater of terminal guanidine groups. In another embodiment, the biguanide composition comprises less than about 18 mole % of terminal amine groups and about 55 mol % or greater of terminal guanidine groups.

In this application, this biguanide composition is referred to as PHMB-CG*. Polymeric biguanide compositions are also referred to in the generic sense as “hexamethylene biguanides”, which one of ordinary skill in the art would recognize to include both PHMB as well as PHMB-CG*.

Representative examples of suitable quaternary ammonium compounds for use in the ophthalmic compositions of the present invention include, but are not limited to, poly[(dimethyliminio)-2-butene-1,4-diyl chloride] and [4-tris(2-hydroxyethyl)ammonio]-2-butenyl-w-[tris(2-hydroxyethyl)ammonio]-dichloride (chemical registry no. 75345-27-6) generally available as Polyquaternium 1 under the tradename ONAMER® M (Stepan Company, Northfield, Ill), and the like and mixtures thereof.

Suitable terpene antimicrobial agents for use in the ophthalmic compositions of the present invention include any monoterpene, sesquiterpene and/or diterpene or derivatives thereof. Acyclic, monocyclic and/or bicyclic mono-, sesqui- and/or diterpenes, and those with higher numbers of rings, can be used. A “derivative” of a terpene as used herein shall be understood to mean a terpene hydrocarbon having one or more functional groups such as terpene alcohols, terpene ethers, terpene esters, terpene aldehydes, terpene ketones and the like and combinations thereof. Here, both the trans and also the cis isomers are suitable. The terpenes as well as the terpene moiety in the derivative can contain from 6 to about 100 carbon atoms and preferably from about 10 to about 25 carbon atoms.

Representative examples of suitable terpene alcohol antimicrobial agents include verbenol, transpinocarveol, cis-2-pinanol, nopol, isoborneol, carbeol, piperitol, thymol, α-terpineol, terpinen-4-ol, menthol, 1,8-terpin, dihydro-terpineol, nerol, geraniol, linalool, citronellol, hydroxycitronellol, 3,7-dimethyl octanol, dihydro-myrcenol, tetrahydro-alloocimenol, perillalcohol, falcarindiol and the like and mixtures thereof.

Representative examples of suitable terpene ether and terpene ester antimicrobial agents include 1,8-cineole, 1,4-cineole, isobornyl methylether, rose pyran, α-terpinyl methyl ether, menthofuran, trans-anethole, methyl chavicol, allocimene diepoxide, limonene mono-epoxide, isobornyl acetate, nonyl acetate, α-terpinyl acetate, linalyl acetate, geranyl acetate, citronellyl acetate, dihydro-terpinyl acetate, meryl acetate and the like and mixtures thereof.

Representative examples of terpene aldehyde and terpene ketone antimicrobial agents include myrtenal, campholenic aldehyde, perillaldehyde, citronellal, citral, hydroxy citronellal, camphor, verbenone, carvenone, dihydro-carvone, carvone, piperitone, menthone, geranyl acetone, pseudo-ionone, a-ionine, iso-pseudo-methyl ionone, n-pseudo-methyl ionone, iso-methyl ionone, n-methyl ionone and the like and mixtures thereof. Any other terpene hydrocarbons having functional groups known in the art may be used herein in the inventive composition.

In one illustrative embodiment, suitable terpenes or derivatives thereof as antimicrobial agents include, but are not limited to, tricyclene, α-pinene, terpinolene, carveol, amyl alcohol, nerol, β-santalol, citral, pinene, nerol, b-ionone, caryophillen (from cloves), guaiol, anisaldehyde, cedrol, linalool, d-limonene (orange oil, lemon oil), longifolene, anisyl alcohol, patchouli alcohol, α-cadinene, 1,8-cineole, p-cymene, 3-carene, ρ-8-mentane, trans-menthone, borneol, α-fenchol, isoamyl acetate, terpin, cinnamic aldehyde, ionone, geraniol (from roses and other flowers), myrcene (from bayberry wax, oil of bay and verbena), nerol, citronellol, carvacrol, eugenol, carvone, α-terpineol, anethole, camphor, menthol, limonene, nerolidol, farnesol, phytol, carotene (vitamin A₁), squalene, thymol, tocotrienol, perillyl alcohol, borneol, simene, carene, terpenene, linalool, 1-terpene-4-ol, zingiberene (from ginger) and the like and mixtures thereof.

In one illustrative embodiment, the compound of component (ii) of the ophthalmic composition comprises a branched, glycerol monoalkyl ether. In another embodiment, the compound of component (ii) of the ophthalmic composition comprises a branched, glycerol monoalkyl amine. In another embodiment, the compound of component (ii) of the ophthalmic composition comprises a branched, glycerol monoalkyl sulphide. In still another embodiment, the compound of component (ii) of the ophthalmic composition comprises any one mixture of a branched, glycerol monoalkyl ether, a branched, glycerol monoalkyl amine or a branched, glycerol monoalkyl sulphide.

In one illustrative embodiment, the branched, glycerol monoalkyl ether for use in the ophthalmic compositions of the present invention is 3-[(2-ethylhexyl)oxy]-1,2-propanediol (EHOPD). In another embodiment, the branched, glycerol monoalkyl amine is 3-[(2-ethylhexyl)amino]-1,2-propanediol (EHAPD). In another embodiment, the branched, glycerol monoalkyl sulphide is 3-[(2-ethylhexyl)thio]-1,2-propanediol (EHSPD). In still another embodiment, the ophthalmic composition comprises any one mixture of EHOPD, EHAPD and EHSPD. The chemical structures of EHOPD, EHAPD and EHSPD are provided below.

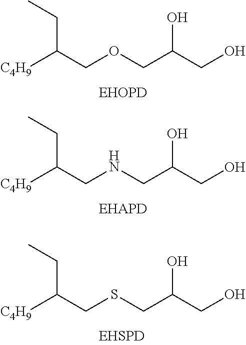

EHOPD is also referred to as octoxyglycerin and is sold under the tradename Sensiva® SC50 (Schülke & Mayr). EHOPD is a branched, glycerol monoalkyl ether known to be gentle to the skin, and to exhibit antimicrobial activity against a variety of Gram-positive bacteria such as Micrococcus luteus, Corynebacterium aquaticum, Corynebacterium flavescens, Corynebacterium callunae, and Corynebacterium nephredi. Accordingly, EHOPD is used in various skin deodorant preparations at concentrations between about 0.2 and 3 percent by weight. EHAPD can be prepared from 2-ethylhexylamine and 2,3-epoxy-1-propanediol using chemistry well known to those of ordinary skill in the art. EHSPD can be prepared from 2-ethylhexylthiol and 2,3-epoxy-1-propanediol using chemistry well known to those of ordinary skill in the art.

Suitable fatty acid monoester for use in the ophthalmic compositions of the present invention include those fatty acid monoesters comprising an aliphatic fatty acid portion having six to fourteen carbon atoms, and an aliphatic hydroxyl portion.

The term “aliphatic” refers to a straight or branched, saturated or unsaturated hydrocarbon having six to fourteen carbon atoms. In one embodiment, the aliphatic fatty acid portion is a straight chain, saturated or unsaturated hydrocarbon with eight to ten carbons. In another embodiment, the aliphatic fatty acid portion is a branched chain, saturated or unsaturated hydrocarbon with eight to ten carbons.

The aliphatic hydroxyl portion of the fatty acid monoester can be any aliphatic compound with at least one hydroxyl group. In many of the embodiments, the aliphatic hydroxyl portion will have from three to nine carbons. The aliphatic hydroxyl portion can include, but is not limited to, propylene glycol, glycerol, a polyalkylene glycol, e.g., polyethylene glycol or polypropylene glycol, a cyclic polyol, e.g., sorbitan, glucose, mannose, sucrose, fructose, fucose and inisitol and derivatives thereof, and a linear polyol, e.g., mannitol and sorbitol and derivatives thereof and the like and mixtures thereof.

Representative examples of suitable amidoamines for use in the ophthalmic compositions of the present inventions include those amidoamines of the general formula: R¹⁵—(OCH₂CH₂)ₘ—X—(CH₂)ₙ—Y wherein R¹⁵ is a is C₆-C₃₀ saturated or unsaturated hydrocarbon including by way of example, a straight or branched, substituted or unsubstituted alkyl, alkylaryl, or alkoxyaryl group; m is zero to 16; n is 2 to 16; X is —C(O)—NR¹⁶— or —R¹⁶N—C(O)—; Y is —N(R¹⁷)₂ wherein each of R¹⁶ and R¹⁷ independently are hydrogen, a C₁-C₈ saturated or unsaturated alkyl or hydroxyalkyl, or a pharmaceutically acceptable salt thereof.

Some of the amidoamines utilized in the present invention are available from commercial sources. For example, myristamidopropyl dimethylamine is available from Alcon Inc. (Fort Worth, Tx.) under the tradename Aldox®; lauramidopropyl dimethylamine is available from Inolex Chemical Company (Philadelphia, Pa.) under the tradename LEXAMINE® L-13; and stearamidopropyl dimethylamine is also from Inolex Chemical Company as LEXAMINE® S-13. The above-described amidoamines can be synthesized in accordance with known techniques, including those described in U.S. Pat. No. 5,573,726.

The amount of the primary antimicrobial agent may vary depending on the specific agent employed. For the aforementioned organic nitrogen-containing agent, typically, such agents are present in concentrations ranging from about 0.00001 to about 0.5 wt. %, or from about 0.00003 to about 0.05 wt. %. For sorbic acid, higher amounts may be required, typically about 0.01 to about 1 wt. %, or from about 0.1 to about 0.5 wt. %. It is preferred that the antimicrobial agent is used in an amount that will at least partially reduce the microorganism population in the formulations employed. If desired, the antimicrobial agent may be employed in a disinfecting amount, which will reduce the microbial bioburden by at least two log orders in four hours and more preferably by one log order in one hour. Most preferably, a disinfecting amount is an amount which will eliminate the microbial burden on a contact lens when used in regimen for the recommended soaking time (FDA Chemical Disinfection Efficacy Test-July, 1985 Contact Lens Solution Draft Guidelines).

The aqueous ophthalmic solutions may further contain one or more other components that are commonly present in ophthalmic solutions, for example, surfactants, tonicity adjusting agents; buffering agents; chelating agents; pH adjusting agents, viscosity modifying agents, and demulcents and the like as discussed hereinabove, and which aid in making aqueous ophthalmic solutions more comfortable to the user and/or more effective for their intended use.

In some embodiments, the pH of the aqueous ophthalmic solutions may be maintained within the range of pH of about 4.0 to about 9.0, or about 5.0 to about 8.0, or about 6.0 to about 8.0, or about 6.5 to about 7.8. In one embodiment, pH values of greater than or equal to about 7 at most.

The following examples are provided to enable one skilled in the art to practice the invention and are merely illustrative. The examples should not be read as limiting the scope of the invention as defined in the claims.

### Example 1

Preparation of oleyl amine sulfobetaine by the following the general reaction scheme.

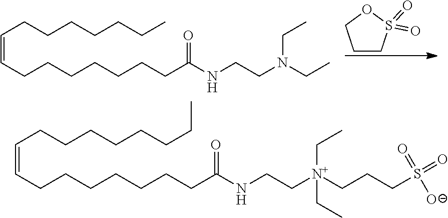

A two-neck round bottom flask fitted with magnetic stir bar, reflux condenser, and thermocouple was charged with 1.021 g (2.682 mmol, 1 eq) amine and 0.521 g (4.266 mmol, 1.6 eq) 1,3-propanesultone. MeCN was added (9 mL) and the reaction mixture was heated to temperature setpoint of 82 °C. Initially the amine was immiscible in the reaction mixture, until the internal temperature reached 40 °C, at which point the reaction mixture became homogeneous. The reaction mixture was heated at reflux overnight and allowed to cool to room temperature, at which point the reaction product precipitated as a waxy tannish-orange material. The MeCN was decanted, the flask was re-charged with fresh MeCN, and the flask heated to boiling to homogenize. The reaction mixture was allowed to cool to room temperature, again precipitating a waxy material. The MeCN was decanted, and for a second time the flask was re-charged with fresh MeCN, and the flask heated to boiling to homogenize. The reaction mixture was allowed to cool to room temperature, precipitating a waxy tannish-orange material. The waxy product was transferred to a tared vial and heated gently under vacuum (approximately 4 mbar) to produce 0.62 g (1.2 mmol) final product in moderate yield (45%), although yield is expected to improve upon optimization. The reaction product was confirmed by H-NMR (400 MHz) in DMSO-d6.

### Example 2

Ten Kalifilcon A contact lenses were soaked in an IPA solution of 0.5 g of the oleyl amine sulfobetaine of Example 1 in 50 g IPA for 30 minutes. The contact lenses were transferred to a 0.5% solution obtained by diluting 1:1 of an IPA solution of 0.5 g of the oleyl amine sulfobetaine of Example 1 in 50 g IPA with water for 30 minutes. The contact lenses were rinsed in water and examined under illumination versus non-treated Kalifilcon A contact lenses. There were no qualitative differences in lens clarity or lubricity.

According to an aspect of the present disclosure, a zwitterionic sulfobetaine-containing lipid comprises a zwitterionic sulfobetaine group attached to one or more lipid groups each comprising at least one fatty acid acyl chain.

In non-limiting illustrative embodiments, as may be combined with one or more of the preceding paragraphs, the at least one fatty acid acyl chain comprises from about 6 to about 30 carbon atoms.

In non-limiting illustrative embodiments, as may be combined with one or more of the preceding paragraphs, an acyl moiety of the at least one fatty acid acyl chain comprises one of an ester moiety or an amide moiety.

In non-limiting illustrative embodiments, as may be combined with one or more of the preceding paragraphs, an acyl moiety of the at least one fatty acid acyl chain is attached to the zwitterionic sulfobetaine group by an alkylene group.

In non-limiting illustrative embodiments, as may be combined with one or more of the preceding paragraphs, the one or more lipid groups each comprising at least one fatty acid acyl chain comprise a first lipid group comprising a first fatty acid acyl chain and a second lipid group comprising a second fatty acid acyl chain.

In non-limiting illustrative embodiments, as may be combined with one or more of the preceding paragraphs, the one or more lipid groups each comprising at least one fatty acid acyl chain comprise a lipid group comprising a first fatty acid acyl chain and a second fatty acid acyl chain.

In non-limiting illustrative embodiments, as may be combined with one or more of the preceding paragraphs, the zwitterionic sulfobetaine-containing lipid is represented by the following structure:

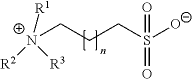

wherein n is 1 or 2, R¹ is a hydrocarbyl group, R² is a hydrocarbyl group or one or more lipid groups each comprising at least one fatty acid acyl chain and R³ is a hydrocarbyl group or one or more lipid groups comprising at least one fatty acid acyl chain, wherein at least one of R² or R³ is one or more lipid groups each comprising at least one fatty acid acyl chain.

In non-limiting illustrative embodiments, as may be combined with one or more of the preceding paragraphs, the at least one fatty acid acyl chain comprises from about 6 to about 30 carbon atoms.

In non-limiting illustrative embodiments, as may be combined with one or more of the preceding paragraphs, an acyl moiety of the at least one fatty acid acyl chain comprises one of an ester moiety or an amide moiety.

In non-limiting illustrative embodiments, as may be combined with one or more of the preceding paragraphs, an acyl moiety of the at least one fatty acid acyl chain is attached to the zwitterionic sulfobetaine group by an alkylene group.

In non-limiting illustrative embodiments, as may be combined with one or more of the preceding paragraphs, R² is a first lipid group comprising a first fatty acid acyl chain and R³ is a second lipid group comprising a second fatty acid acyl chain.

In non-limiting illustrative embodiments, as may be combined with one or more of the preceding paragraphs, R² is an alkyl group and R³ is a lipid group comprising a first fatty acid acyl chain and a second fatty acid acyl chain.

In non-limiting illustrative embodiments, as may be combined with one or more of the preceding paragraphs, the zwitterionic sulfobetaine-containing lipid is represented by a structure of Formula I:

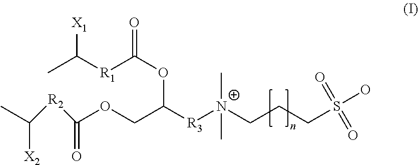

wherein R₁ is a saturated or unsaturated linear diradical C₁₃ to C₂₇ hydrocarbon chain; R₂ is a saturated or unsaturated linear diradical C₁₃ to C₂₇ hydrocarbon chain; R₃ is an alkylene group; X₁ and X₂ are independently H or CH₃; and n is 1 or 2, or by a structure of Formula II:

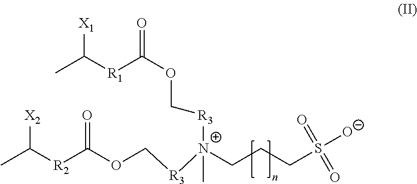

wherein R₁ is a saturated or unsaturated linear diradical C₁₃ to C₂₇ hydrocarbon chain; R₂ is a saturated or unsaturated linear diradical C₁₃ to C₂₇ hydrocarbon chain; R₃ is an alkylene group; X₁ and X₂ are independently H or CH₃; and n is 1 or 2, or by a structure of Formula III:

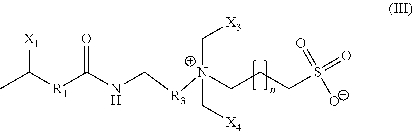

wherein R₁ is a saturated or unsaturated linear diradical C₁₃ to C₂₇ hydrocarbon chain; R₃ is an alkylene group; X₁, X₃, and X₄ are independently H or CH₃; and n is 1 or 2.

In non-limiting illustrative embodiments, as may be combined with one or more of the preceding paragraphs, R₃ is methylene.

According to another aspect of the present disclosure, an ophthalmic device comprises the zwitterionic sulfobetaine-containing lipid according to any one of the preceding paragraphs entrapped in the ophthalmic device.

In non-limiting illustrative embodiments, as may be combined with one or more of the preceding paragraphs, the ophthalmic device is a polymerization product of a monomeric mixture comprising one or more hydrophilic monomers.

In non-limiting illustrative embodiments, as may be combined with one or more of the preceding paragraphs, the monomeric mixture further comprises the zwitterionic sulfobetaine-containing lipid.

In non-limiting illustrative embodiments, as may be combined with one or more of the preceding paragraphs, the ophthalmic device is a contact lens.

In non-limiting illustrative embodiments, as may be combined with one or more of the preceding paragraphs, the ophthalmic device is a polymerization product of a monomeric mixture comprising one or more silicone monomers.

In non-limiting illustrative embodiments, as may be combined with one or more of the preceding paragraphs, the monomeric mixture further comprises the zwitterionic sulfobetaine-containing lipid.

In non-limiting illustrative embodiments, as may be combined with one or more of the preceding paragraphs, the ophthalmic device is a silicone contact lens.

In non-limiting illustrative embodiments, as may be combined with one or more of the preceding paragraphs, the ophthalmic device is a silicone hydrogel.

According to another aspect of the present disclosure, a process for preparing an ophthalmic device having a zwitterionic sulfobetaine-containing lipid entrapped in the ophthalmic device comprises:

subjecting an ophthalmic device-forming mixture comprising one or more ophthalmic device-forming monomers and the zwitterionic sulfobetaine-containing lipid according to any one of the preceding paragraphs, to polymerization conditions to provide a polymerized ophthalmic device, and

hydrating the polymerized ophthalmic device.

In non-limiting illustrative embodiments, as may be combined with one or more of the preceding paragraphs, the one or more ophthalmic device-forming monomers comprise one or more hydrophilic monomers.

In non-limiting illustrative embodiments, as may be combined with one or more of the preceding paragraphs, the ophthalmic device is a contact lens.

In non-limiting illustrative embodiments, as may be combined with one or more of the preceding paragraphs, the one or more ophthalmic device-forming monomers comprise one or more silicone monomers.

In non-limiting illustrative embodiments, as may be combined with one or more of the preceding paragraphs, the ophthalmic device is a silicone contact lens.

In non-limiting illustrative embodiments, as may be combined with one or more of the preceding paragraphs, the silicone contact lens is a silicone hydrogel.

According to another aspect of the present disclosure, a process for preparing an ophthalmic device having a zwitterionic sulfobetaine-containing lipid entrapped in the ophthalmic device comprises:
soaking an ophthalmic device in one or more first solvent solutions to swell the ophthalmic device, thereby providing a swelled ophthalmic device,
soaking the swelled ophthalmic device in one or more second solvent solutions to moieties de-swell the ophthalmic device, thereby providing a de-swelled ophthalmic device, and
sterilizing the de-swelled ophthalmic device,
wherein the one or more first solvent solutions, the one or more second solvent solutions or both comprise the zwitterionic sulfobetaine-containing lipid according to any one the preceding paragraphs.

In non-limiting illustrative embodiments, as may be combined with one or more of the preceding paragraphs, the one or more first solvent solutions comprise one or more of a low molecular weight alcohol solvent, an aliphatic hydrocarbon solvent, a cycloaliphatic hydrocarbon solvent, a ketone solvent, a nitrile solvent, an ether solvent, and an amido group-containing solvent.

In non-limiting illustrative embodiments, as may be combined with one or more of the preceding paragraphs, the one or more first solvent solutions comprise a blend of a low molecular weight alcohol solvent and water.

In non-limiting illustrative embodiments, as may be combined with one or more of the preceding paragraphs, process comprises:
(i) soaking the ophthalmic device in the one or more first solvent solution comprising a blend of a low molecular weight alcohol solvent and water for a time period ranging from about 5 minutes to about 120 minutes, and
(ii) soaking the ophthalmic device of step (i) in another solvent solution comprising the same or different one or more low molecular weight alcohol solvent, an aliphatic hydrocarbon solvent, a cycloaliphatic hydrocarbon solvent, a ketone solvent, a nitrile solvent, an ether solvent, or an amido group-containing solvent for a time period ranging from about 5 minutes to about 60 minutes.

In non-limiting illustrative embodiments, as may be combined with one or more of the preceding paragraphs, step (b) comprises:
(i) soaking the swelled ophthalmic device in the one or more second solvent solutions comprising a low molecular weight alcohol solvent for a time period ranging from about 5 minutes to about 20 minutes, and
(ii) soaking the swelled ophthalmic device of step (i) in another solvent solution comprising a blend of the same or different low molecular weight alcohol solvent of the one or more second solvent solutions and water for a time period ranging from about 5 minutes to about 20 minutes.

In non-limiting illustrative embodiments, as may be combined with one or more of the preceding paragraphs, step (c) comprises autoclaving the ophthalmic device.

In non-limiting illustrative embodiments, as may be combined with one or more of the preceding paragraphs, the process further comprises the step of applying a lid stock to a package containing the ophthalmic device prior to subjecting the ophthalmic device to a step of autoclaving.

In non-limiting illustrative embodiments, as may be combined with one or more of the preceding paragraphs, the ophthalmic device is one or more of a contact lens, an intraocular lens or a corneal implant.

According to another aspect of the present disclosure, a packaging system for the storage of an ophthalmic device is provided comprising a sealed container containing an unused ophthalmic device immersed in an aqueous packaging solution comprising the zwitterionic sulfobetaine-containing lipid according to any one of the preceding paragraphs, wherein the aqueous packaging solution has an osmolality of at least about 200 mOsmol/kg, a pH of about 6 to about 9 and is sterilized.

In non-limiting illustrative embodiments, as may be combined with one or more of the preceding paragraphs, the unused ophthalmic device is an unused contact lens.

According to another aspect of the present disclosure, a method of preparing a package comprising a storable, sterile ophthalmic device comprising:
(a) immersing an unused ophthalmic device in an aqueous packaging solution comprising the zwitterionic sulfobetaine-containing lipid according to any one of the preceding paragraphs, wherein the aqueous packaging solution has an osmolality of at least about 200 mOsmol/kg and a pH in the range of about 6 to about 9,
(b) packaging the aqueous packaging solution and the unused ophthalmic device in a manner preventing contamination of the device by microorganisms, and
(c) sterilizing the packaged aqueous packaging solution and the unused ophthalmic device.

In non-limiting illustrative embodiments, as may be combined with one or more of the preceding paragraphs, the unused ophthalmic device is an unused contact lens.

According to another aspect of the present disclosure, an aqueous ophthalmic composition comprises the zwitterionic sulfobetaine-containing lipid according to any one of the preceding paragraphs, wherein the aqueous ophthalmic composition has an osmolality in a range from about 200 mOsmol/kg to about 400 mOsmol/kg.

In non-limiting illustrative embodiments, as may be combined with one or more of the preceding paragraphs, the aqueous ophthalmic composition is in the form of an eye care or a contact lens care product selected from the group consisting of eye drops, a contact lens preservative solution, a contact lens cleaning solution, and a contact lens multi-purpose solution.

In non-limiting illustrative embodiments, as may be combined with one or more of the preceding paragraphs, the aqueous ophthalmic composition is in the form of a multi-purpose solution or rewetting drops.

In non-limiting illustrative embodiments, as may be combined with one or more of the preceding paragraphs, the aqueous ophthalmic composition is preservative free.

Various features disclosed herein are, for brevity, described in the context of a single embodiment, but may also be provided separately or in any suitable sub-combination. All combinations of the embodiments are specifically embraced by the illustrative embodiments disclosed herein just as if each and every combination was individually and explicitly disclosed. In addition, all sub-combinations listed in the embodiments describing such variables are also specifically embraced by the present compositions and are disclosed herein just as if each and every such sub-combination was individually and explicitly disclosed herein.

It will be understood that various modifications may be made to the embodiments disclosed herein. Therefore, the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention. Moreover, those skilled in the art will envision other modifications within the scope and spirit of the features and advantages appended hereto.

What is claimed is:

1. A zwitterionic sulfobetaine-containing lipid comprising a zwitterionic sulfobetaine group attached to one or more lipid groups each comprising at least one fatty acid acyl chain.

2. The zwitterionic sulfobetaine-containing lipid according to claim 1, wherein the at least one fatty acid acyl chain comprises from about 6 to about 30 carbon atoms.

3. The zwitterionic sulfobetaine-containing lipid according to claim 1, wherein an acyl moiety of the at least one fatty acid acyl chain comprises one of an ester moiety or an amide moiety.

4. The zwitterionic sulfobetaine-containing lipid according to claim 1, wherein an acyl moiety of the at least one fatty acid acyl chain is attached to the zwitterionic sulfobetaine group by an alkylene group.

5. The zwitterionic sulfobetaine-containing lipid according to claim 1, wherein the one or more lipid groups each comprising at least one fatty acid acyl chain comprise a first lipid group comprising a first fatty acid acyl chain and a second lipid group comprising a second fatty acid acyl chain.

6. The zwitterionic sulfobetaine-containing lipid according to claim 1, wherein the one or more lipid groups each comprising at least one fatty acid acyl chain comprise a lipid group comprising a first fatty acid acyl chain and a second fatty acid acyl chain.

7. The zwitterionic sulfobetaine-containing lipid according to claim 1, which is represented by the following structure: 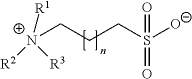
wherein n is 1 or 2, R¹ is a hydrocarbyl group, R² is a hydrocarbyl group or a lipid group comprising at least one fatty acid acyl chain and R³ is a hydrocarbyl group or a lipid group comprising at least one fatty acid acyl chain, wherein at least one of R² or R³ is a lipid group comprising at least one fatty acid acyl chain.

8. The zwitterionic sulfobetaine-containing lipid according to claim 1, which is represented by a structure of Formula I: 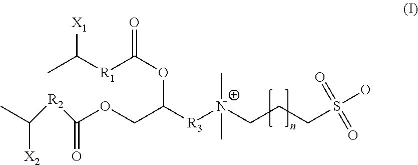
wherein R¹ is a saturated or unsaturated linear diradical C₁₃ to C₂₇ hydrocarbon chain; R₂ is a saturated or unsaturated linear diradical C₁₃ to C₂₇ hydrocarbon chain; R₃ is an alkylene group; X₁ and X₂ are independently H or CH₃; and n is 1 or 2, or by a structure of Formula II: 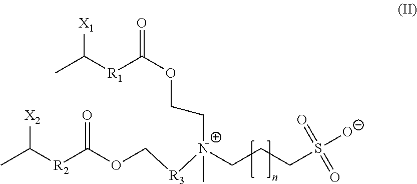
wherein R¹ is a saturated or unsaturated linear diradical C₁₃ to C₂₇ hydrocarbon chain; R₂ is a saturated or unsaturated linear diradical C₁₃ to C₂₇ hydrocarbon chain; R₃ is an alkylene group; X₁ and X₂ are independently H or CH₃; and n is 1 or 2, or by a structure of Formula III: 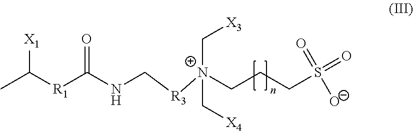
wherein R₁ is a saturated or unsaturated linear diradical C₁₃ to C₂₇ hydrocarbon chain; R₃ is an alkylene group; X₁, X₃, and X₄ are independently H or CH₃; and n is 1 or 2.

9. The zwitterionic sulfobetaine-containing lipid according to claim 8, wherein R₃ of the structure of Formula I, II or III is methylene.

10. An ophthalmic device comprising a zwitterionic sulfobetaine-containing lipid comprising a zwitterionic sulfobetaine group attached to one or more lipid groups each comprising at least one fatty acid acyl chain, wherein the zwitterionic sulfobetaine-containing lipid is entrapped in the ophthalmic device.

11. The ophthalmic device according to claim 10, wherein the ophthalmic device is a polymerization product of a monomeric mixture comprising the zwitterionic sulfobetaine-containing lipid.

12. The ophthalmic device according to claim 11, wherein the monomeric mixture further comprises one or more hydrophilic monomers, one or more silicone monomers, or both.

13. The ophthalmic device according to claim 10, wherein the zwitterionic sulfobetaine-containing lipid is represented by the following structure: 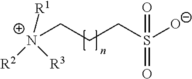
wherein n is 1 or 2, R¹ is a hydrocarbyl group, R² is a hydrocarbyl group or a lipid group comprising at least one fatty acid acyl chain and R³ is a hydrocarbyl group or a lipid group comprising at least one fatty acid acyl chain, wherein at least one of R² or R³ is a lipid group comprising at least one fatty acid acyl chain.

14. The ophthalmic device according to claim 10, wherein the zwitterionic sulfobetaine-containing lipid is represented by a structure of Formula I: 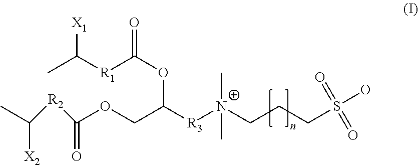
wherein R₁ is a saturated or unsaturated linear diradical C₁₃ to C₂₇ hydrocarbon chain; R₂ is a saturated or unsaturated linear diradical C₁₃ to C₂₇ hydrocarbon chain; R₃ is an alkylene group; X₁ and X₂ are independently H or CH₃; and n is 1 or 2, or by a structure of Formula II: 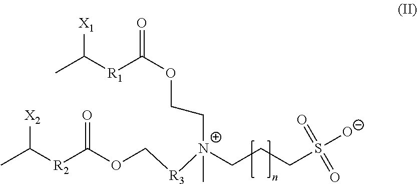
wherein R₁ is a saturated or unsaturated linear diradical C₁₃ to C₂₇ hydrocarbon chain; R₂ is a saturated or unsaturated linear diradical C₁₃ to C₂₇ hydrocarbon chain; R₃ is an alkylene group; X₁ and X₂ are independently H or CH₃; and n is 1 or 2, or by a structure of Formula III: 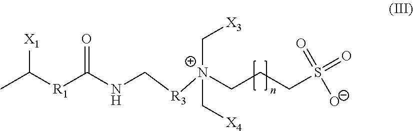
wherein R₁ is a saturated or unsaturated linear diradical C₁₃ to C₂₇ hydrocarbon chain; R₃ is an alkylene group; X₁, X₃, and X₄ are independently H or CH₃; and n is 1 or 2.

15. The ophthalmic device according to claim 10, which is a contact lens.

16. A process for preparing an ophthalmic device having a zwitterionic sulfobetaine-containing lipid entrapped in the ophthalmic device, the process comprising:
subjecting an ophthalmic device-forming mixture comprising one or more ophthalmic device-forming monomers and a zwitterionic sulfobetaine-containing lipid comprising a zwitterionic sulfobetaine group attached to one or more lipid groups each comprising at least one fatty acid acyl chain to polymerization conditions to provide a polymerized ophthalmic device; and
hydrating the polymerized ophthalmic device.

17. The process according to claim 16, wherein the one or more ophthalmic device-forming monomers comprise one or more hydrophilic monomers, one or more silicone monomers, or both.

18. The process according to claim 16, wherein the ophthalmic device is a contact lens.

19. A process for preparing an ophthalmic device having a zwitterionic sulfobetaine-containing lipid entrapped in the ophthalmic device, the process comprising:
soaking an ophthalmic device in one or more first solvent solutions to swell the ophthalmic device, thereby providing a swelled ophthalmic device;
soaking the swelled ophthalmic device in one or more second solvent solutions to moieties de-swell the ophthalmic device, thereby providing a de-swelled ophthalmic device; and
sterilizing the de-swelled ophthalmic device;
wherein the one or more first solvent solutions, the one or more second solvent solutions or both comprise the zwitterionic sulfobetaine-containing lipid according to claim 1.

20. The process according to claim 19, wherein the ophthalmic device is a contact lens.
